(19) United States
(12) Patent Application Publication
(10) Pub. No.: US 2010/0143241 A1
(43) Pub. Date: Jun. 10, 2010

(54) Method and apparatus for delivery of agents across the blood brain barrier

(76) Inventors: G. Allan Johnson, Durham, NC (US); Gabriel Howles-Banerji, Durham, NC (US); Kristin Frinkley Bing, Atlanta, GA (US); Kathryn Nightingale, Durham, NC (US); Mark Palmeri, Durham, NC (US)

Correspondence Address: NIXON & VANDERHYE, PC, 901 NORTH GLEBE ROAD, 11TH FLOOR, ARLINGTON, VA, 22203, US

(21) Appl. No.: 12/588,667

(22) Filed: Oct. 22, 2009

Related U.S. Application Data
(60) Provisional application No. 61/193,006, filed on Oct. 22, 2008.

(51) Int. Cl. A61K 51/00; A61B 8/00; A61K 49/06; A61K 49/04; A61K 49/22

(52) U.S. Cl. 424/1.11; 424/9.5; 424/9.3; 424/9.4

(57) ABSTRACT

We describe a method for opening the blood-brain barrier (BBB) using ultrasound and preformed microbubbles. With this method, diagnostic or therapeutic agents may be administered to the brain. This method can open a focal region of the BBB and administer agents in a targeted fashion or the method can open large regions (or the entirety) of the brain for more global administration of agents. In one embodiment, the method can be used to administer contrast agents (e.g., agents that increase or decrease the magnetic resonance imaging signal) to the brain and thereby improve the quality or information content of imaging data. In another embodiment, a standard clinical diagnostic ultrasound scanner can be used to open specific regions of the BBB and administer diagnostic or therapeutic agents. Importantly, this invention can open the BBB in a non-destructive/non-invasive fashion, allowing the subject to be awake and suffer no detectable side effects.

### RELATED APPLICATIONS

This Application claims the benefit of priority from U.S. Provisional Application Ser. No. 61/193,006 filed on Oct. 22, 2008. This provisional application is hereby incorporated in its entirety by reference.

### FEDERALLY-SPONSORED RESEARCH OR DEVELOPMENT

The U.S. Government has certain rights in this invention as provided for by the terms of NIH/NCRR: 5P41-RR005959-18, 2U24CA092656-07, 5R01CA114075, 2R01EB002132-05 and NSF 2003014921 awarded by the Department of Health and Human Services.

### BACKGROUND OF THE INVENTION

### Active Staining

In magnetic resonance imaging, the term “active staining” refers to the use of contrast agents to selectively enhance specific tissue properties to improve the MRI images. Active staining was introduced by the Center for In Vivo Microscopy in 2002 for perfusion-fixed specimens (Johnson 2002). A patent was subsequently issued for active staining of fixed specimens (U.S. Pat. No. 6,023,162). Contrast agents are routinely used in clinical MRI to enhance or otherwise alter the signal. The majority of the contrast agents that enhance signal do so by reducing the spin-lattice relaxation time (T1), usually by coupling the protons in the tissue to unpaired electrons in the magnetic resonance contrast agents. Other agents alter the signal through reduction of spin-spin relaxation time (T2). However, none of these contrast agents readily penetrate the blood brain barrier. The intact blood-brain barrier (BBB) in the live animal has heretofore made it challenging to perform active staining in vivo.

### Blood-Brain Barrier Disruption

In humans and animals, blood-brain barrier disruption (BBBD) in a single hemisphere of the brain is typically performed by intracarotid infusion of a hypertonic solution of arabinose or mannitol (Kroll 1998). In rodents, the preferred procedure involves surgical placement of a catheter in the external carotid artery. In small rodents such mice, the placement of the catheter is extremely difficult. An alternative method is to inject the hypertonic solution by way of the more accessible common carotid (Deng, 1998), but this necessarily disrupts blood flow to the brain, creating a perilous confound for most experiments. The invasiveness of both methods makes them unsuitable for survival studies and the technical difficulty makes the methods unsuitable for routine use in mice. Furthermore, the physiological effects of the hypertonic solution may confound scientific studies. Finally, these techniques open the blood-brain barrier in only one hemisphere of the brain.

The use of an MRI contrast agent with disruption of the blood brain barrier through the injection of hypertonic solution has been demonstrated by several groups. However as noted above, this method has serious technical and scientific limitation, particularly when applied to mice.

Localized blood-brain barrier disruption in small animals has been performed by a few groups using focused ultrasound combined with microbubble ultrasound contrast agents, such as Optison (FS069) and Definity (perflutren lipid microsphere, Lantheus Medical Imaging, North Billerica, Mass.) (Hynynen 2001, Mesiwala 2002, Choi 2007, McDannold 2007). Focused ultrasound is capable of producing very high pressure, which can produce neuronal damage; however, it has been demonstrated that blood-brain barrier disruption can be achieved using lower pressures that do not cause damage that can be observed with conventional histology (Hynynen 2001, Mesiwala 2002). While the precise mechanism of blood-brain barrier disruption is not known, current data suggests it is neither cavitation nor a thermal effect (McDannold 2006, Sheikov 2004). This focused ultrasound technique has be used to administer contrast agents to the localized regions in the brain in animals.

All patents, patent applications and references cited anywhere in this disclosure are incorporated by reference in their entirety.

### BRIEF DESCRIPTION OF THE INVENTION

The use of diagnostic and therapeutic agents in the brain is limited by the blood-brain barrier (BBB), which restricts entry into the brain. To administer agents to the brain of rats, intracarotid infusions of hypertonic mannitol have been used to open the BBB. However, this technically challenging approach is invasive, opens only a limited region of the BBB, and is difficult to extend to mice. In this work, the BBB was opened in mice using unfocused ultrasound combined with an injection of microbubbles. This technique has several notable features: it (a) can be performed trans-cranially in mice; (b) takes only 3 minutes and uses only commercially available components; (c) opens the BBB throughout the brain, or, if an electronically focused ultrasound beam is used, opens a limited portion of the BBB; (d) causes no observed histological damage or changes in behavior (with peak-negative acoustic pressures of 0.36 MPa); and (e) allows recovery of the BBB within 4 hours. Using this technique, Gd-DTPA was administered to the mouse brain parenchyma, thereby shortening T1 and enabling the acquisition of high-resolution (52×52×100 micrometer3) images in 51 minutes in vivo. By enabling the administration of both existing anatomical contrast agents and the newer molecular/sensing contrast agents, this technique may be useful for the study of mouse models of neurological function and pathology with MRI.

One embodiment of the invention is directed to a method of opening a blood-brain barrier of a subject. The method involves the steps of (a) administering a microbubble agent into the bloodstream of said subject, and (b) applying either (i) an unfocused ultrasound to the whole brain of said subject to open the blood brain barrier in the whole brain, or (ii) an electronically focused ultrasound beam to open a limited portion of the blood brain barrier. In the method, step (a) is performed before step (b) or at the same time as step (b). Preferably, step (a) is performed within 15 minutes, within 10 minutes, within 5 minutes, within 3 minutes or within one minute of step (b) or at the same time as step (b).

The method of the invention is effective for any subject including any animals including humans, primates, livestock, rodents, mice, rats, rabbits, birds, and the like including adult, juvenile or younger, neonatal, and embryonic forms of these animals. Because the techniques are physical rather than pharmacological, it can be used in a variety of species including mammals and non-mammalian species.

The microbubble agent can be any agent known in the art including lipid-type microspheres or protein-type microspheres or a combination thereof in an injectable suspension. For example, the agent can be selected from the group consisting of Octafluoropropane/Albumin (Optison), a perflutren lipid microsphere (Definity), Galactose-Palmitic Acid microbubble suspension (Levovist) Air/Albumin (Albunex and Quantison), Air/Palmitic acid (Levovist/SHU508A), Perfluoropropane/Phospholipids (MRX115, DMP115), Dodecafluoropentane/Surfactant (Echogen/QW3600), Perfluorobutane/Albumin (Perfluorocarbon exposed sonicated dextrose albumin), Perfluorocarbon/Surfactant (QW7437), Perfluorohexane/Surfactant (Imagent/AF0150), Sulphur hexafluoride/Phospholipids (Sonovue/BR1), Perfluorobutane/Phospholipids (BR14), Air/Cyanoacrylate (Sonavist/SHU563A), and Perfluorocarbon/Surfactant (Sonazoid/NC100100).

In a preferred embodiment, the method may be used to administer a therapeutic agent or a diagnostic agent or a combination thereof to the brain or central nervous system of a subject. In that case, the therapeutic agent is administered before step (b) or within 4 hours of step (b). The therapeutic agent may be any agent suitable for administration to the brain or central nervous system including chemotherapeutic agent or a neurotherapeutic agent. Chemotherapeutic agents include any agents known to be therapeutic against cancers including brain cancers and cancers that have metastasized to the brain. Neurotherapeutic agents include, for example, PDGF, VEGF, dopamine and any agent known to be therapeutic to neurological diseases such as Alzheimer's disease, Parkinson disease, stroke, and the like.

The applying step, for the delivery of ultrasound, may comprise the delivery of ultrasound from an ultrasound source through a fluid coupler applied directly to the head of the subject. In this application, the fluid coupler may be applied to only one side or aspect of the subject's head. The head may be an unmodified head or a head with a surgically created window in the skull—the fluid coupler being in contact with the window. The ultrasound may be generated by an unfocused ultrasound transducer or a phased array ultrasound transducer (i.e., focused ultrasound). Significantly, the phased array ultrasound transducer may be a diagnostic phased array. Diagnostic phased arrays are generally of lower power and are commonly available.

For any of the method or apparatus of the invention, the ultrasound transducer may have an output frequency of between 0.1 to 10 MHz. The ultrasound may be applied for a time between 10 milliseconds to 10 minutes. The ultrasound may be applied continuously or in a burst mode. Burst mode may involve a burst mode repetition frequency of between 10 Hz to 100 kHz and burst lengths of 2 microseconds to 100 milliseconds. The fluid coupler may comprise a contained volume of fluid (e.g., about 50 cc, about 100 cc, about 200 cc, about 400 cc, about 500 cc, about 600 cc or about 1 liter). The fluid may be, for example, water, ultrasonic gel, or a substance of comparable acoustic impedance. The fluid may be contained in a fluid cylinder with at least a flexible end portion that conforms to the subject's head. In other embodiments, the contained volume of fluid may be a flexible or elastic fluid container.

Another embodiment of the invention involves providing an imaging contrast agent to the whole brain comprising the steps of (a) administering a microbubble agent into the bloodstream of said subject; (b) administering an imaging contrast agent into the bloodstream of said subject; and (c) applying an unfocused ultrasound to the whole brain of said subject to open the blood brain barrier to allow the image contrast agent to cross the blood brain barrier, wherein step (b) is performed before said steps (a), before said step (c) or within 4 hours after said step (c). Surprisingly, we found that the BBB remains open four hours after treatment with the methods of the invention. Therefore, in a preferred embodiment, any of the agents described in this Specification may be administered to the bloodstream between 1 to 4 hours, between 2 to 4 hours or between 3-4 hours after ultrasound treatment using one of the methods of the invention. This administration has significant benefits because ultrasound delivery and administration may be separated, performed, for example, in different parts of a hospital. Thus, for example, initial ultrasound may be administered in a less than sterile environment such as a hospital room, an ambulance etc and the final delivery may be performed in a clean room. Also, it is possible to administer the ultrasound before an operation (i.e., surgical procedure) and have the BBB open during the operation or during part of the operation. The methods of this invention is not taught or suggested by any of the current methods.

Image contrast agents, used in any methods of the invention, may be selected from the group consisting magnetic resonance contrast agents, x-ray contrast agents (and x-ray computed tomography), optical contrast agents, positron emission tomography (PET) contrast agents, single photon emission computer tomography (SPECT) contrast agents, or molecular imaging agents. For example, the imaging contrast agent may be selected from the group consisting of gadopentetate dimeglumine, Gadodiamide, Gadoteridol, gadobenate dimeglumine, gadoversetamide, iopromide, Iopamidol, Ioversol, or Iodixanol, and Iobitridol.

Another embodiment of the invention involves a method of performing magnetic resonance imaging on a subject comprising the steps of (a) administering a magnetic resonance contrast agent to a subject through the BBB using any of the methods of the invention and performing magnetic resonance, imaging on said subject.

For any of the methods of the invention, the agent, such as the therapeutic agent(s), may be administered to the subject to cross the blood brain barrier to treat the patient. Administration may be, for example, into a blood vessel of a patient such as a vein. Administration may also be any form of injection such as intraperitoneal or intramuscular injection depending the mechanism of drug (agent) transport once it is inside the body. Crossing the BBB may be performed by the method of the invention (e.g., BOMUS). For example, the therapeutic agent may be added to the method of performing MRI. The administration of an imaging contrast agent across the BBB can accompany the administration of a therapeutic agent. Furthermore, by monitoring the brain or any other organ by MRI, the amount of therapeutic agent may be adjusted immediately or in subsequent administration to optimize the dosage, therapeutic level. For example, such adjustment may be valuable for dopamine treatment where excessive dosages would lead to dopamine resistance. Thus, the titration of the dose of such therapeutic agents delivered to the brain through measurement of the change in relaxation times (T1-spin lattice relaxation or T2-spin spin relaxation) by relating the concentration of active stain (MR relaxation agent or other imaging contrast agent) to the concentration of the therapeutic agent may be performed.

For any of the methods of the invention, the ultrasound is optionally delivered only to the brain, or only to the head of the subject. That is, the body of the subject does not receive more than 50%, more than 40%, more than 30%, more than 20% or more than 10% of the total ultrasonic energy to the subject.

Another embodiment of the invention is directed to an apparatus for increasing the permeability of the blood brain barrier in a subject, comprising: an ultrasound emitting device consisting of an ultrasound transducer with appropriate signal generation and amplification, and a fluid coupler for transmitting the ultrasonic output and a microbubble agent. The ultrasound emitting device of the apparatus may use an unfocused ultrasound transducer or an array of unfocused transducers or a phased array ultrasound transducer (i.e., focused ultrasound). The fluid coupler of this apparatus may be any of the fluid coupler described in this disclosure.

### BRIEF DESCRIPTION OF THE DRAWINGS

FIG. 1 depicts the physical arrangement for opening the BBB of a mouse. The components shown in FIG. 1 is as follows: 1) IV injection of a microbubble (ultrasound) contrast agent; 2) IP or IV injection of an MRI contrast agent; 3) ultrasound transducer; 4) fluid cylinder to couple ultrasound into the animal (or patient); 5) ultrasound signal and power generator.

FIG. 2 depicts scans of a) an unstained mouse brain; b) the mouse with gadopentetate dimeglumine c) the mouse with gadopentetate dimeglumine and Definity and d) the actively stained brain show nearly 5× increase in signal from the process of active staining.

FIG. 3 depicts adjacent 100 micron slices of a live mouse brain with 50 micron in-plane resolution acquired at 7T using active in vivo staining with BOMUS and Gd-DTPA.

FIG. 4 depicts images showing a) B-mode ultrasound only (5.7 MHz), b) magnetic resonance only, and c) structures seen in ultrasound (found by thresholding) overlaid in red on the magnetic resonance image.

FIG. 5 depicts a) magnetic resonance image of live mouse prior to active staining using BOMUS. b) magnetic resonance image of the same animal after active staining using focused ultrasound.

FIG. 6 depicts blood-brain barrier opening (performed with Definity and a diagnostic clinical scanner on a mouse) as a function of Definity dose.

FIG. 7 depicts magnetic resonance images of live mice demonstrating BBB opening using Definity and a diagnostic phased array scanner using ultrasound at 4 different frequencies.

FIG. 8 depicts results showing blood-brain barrier opening using Definity and a diagnostic phased array scanner for ultrasonic frequencies from 5 to 8 MHz for the same input voltage.

FIG. 9 depicts magnetic resonance images of live mice demonstrating BBB opening using Definity and a diagnostic phased array scanner using at varied power levels. Note that below 2% of the peak power there is no opening of the blood-brain barrier.

FIG. 10 depicts the effect of ultrasonic pressures (peak to peak) from 1.05 to 6.16 MPa (non-derated) on blood-brain barrier opening using Definity and a diagnostic phased array scanner.

FIG. 11 FIG. 11a depicts the effect of pulse durations on blood-brain barrier opening. FIG. 11b depicts the same data presented as a function of the total number of cycles in the insonification sequence.

FIG. 12 demonstrates opening of the BBB over time as measured by contrast enhancement (normalized to muscle).

FIG. 13 shows magnetic resonance images (top) of a) animal exposed to ultrasound at power levels used for BOMUS and b) at higher power levels. Histology (bottom) of c) the animal submitted to BOMUS shows no tissue damage while d) the histology from the animal exposed to higher levels shows clear evidence of hemorrhage.

FIG. 14 depicts a: The BOMUS setup. b: Experimental time line for active staining with Gd-DTPA using BOMUS.

FIG. 15 depicts the acoustic output of the ultrasound system was characterized in water using a hydrophone. Panel A: The test pulse was a 10-cycle sinusoid (PRF=10 Hz). An input voltage of 167 mVpp produced a peak negative pressure of 0.36 MPa. Panel b: The lateral profile of the beam was measured at the transducers natural focus (58 mm).

FIG. 16 depicts T1-weighted SPGR MR images demonstrating that Gd-DTPA enhances body tissues but is excluded from the brain by the intact BBB unless the BOMUS procedure is performed.

FIG. 17 depicts a time-course of Gd-DTPA enhancement in the brain and muscle after BOMUS.

FIG. 18 depicts the duration of BBB disruption was demonstrated by assaying BBB permeability at several times after BOMUS.

FIG. 19 Panel a depicts the mean number of red blood cell extravasations seen in each histology slide of the brain is shown for peak-negative acoustic pressures of 0.36 MPa (n=3), 0.52 MPa (n=4), and 5.0 MPa (n=1). Error bars show standard error. Panel b depicts an example of severe red blood cell extravasation from the brain exposed to 5.0 MPa.

FIG. 20 depicts overall behavior score before anesthesia and 3 and 24 hours after recovery from anesthesia, demonstrating no detectable adverse affects from the BOMUS procedure at with peak-negative acoustic pressures of 0.36 MPa.

FIG. 21 depicts T1 values from three ROIs in control and treated mice (1 mouse per group).

FIG. 22 High-resolution (52×52×100 mircometer3) SPGR images of the mouse brain acquired in vivo in 51 minutes.

FIG. 23 Minimum intensity projections of a 600-micrometer axial slab from SPGR images from BOMUS-treated mice given high doses of Gd-DTPA demonstrate how active staining can be combined with susceptibility imaging to yield high resolution venograms.

FIG. 24 depicts Example waveforms (a,c) and power spectra (b,d) of pulses with peak-to-peak pressures of 2.72 MPa (a,b) and 6.16 MPa (c,d).

FIG. 25 depicts (a) Anatomical sketch of a coronal slice of the brain with the insonification spots (b) Setup and transducer orientation relative to the mouse. Note: The water bag is not shown here.

FIG. 26 depicts BBB opening with PW Doppler.

FIG. 27 depicts images showing a) B-mode ultrasound only (5.7 MHz), b) MR only, and c) structures seen in ultrasound (found by thresholding) overlaid in red on the MR image.

FIG. 28 depicts BBB opening for ultrasonic transmission frequencies from 5 to 8 MHz for the same system input voltage.

FIG. 29 depicts effect of ultrasonic pressures from 1.05 to 6.16 MPaₚₚ (non-derated) on BBB opening.

FIG. 30 depicts a) Effect of pulse durations of 0.35 μs (B-mode), 2 μs (Color Doppler), 70 μs (Acoustic Radiation Force Impulse Imaging), and 20 ms on BBB opening.

FIG. 31 depicts H & E stained histology of a) blood cell extravasation caused by standard B-mode as well as b) extravasated (top) and vesselenclosed (bottom) blood cells and c) no damage with the most aggressive experimental ultrasound exposure used for this study.

FIG. 32 depicts example of image guidance and system settings for PW Doppler mode BBB opening.

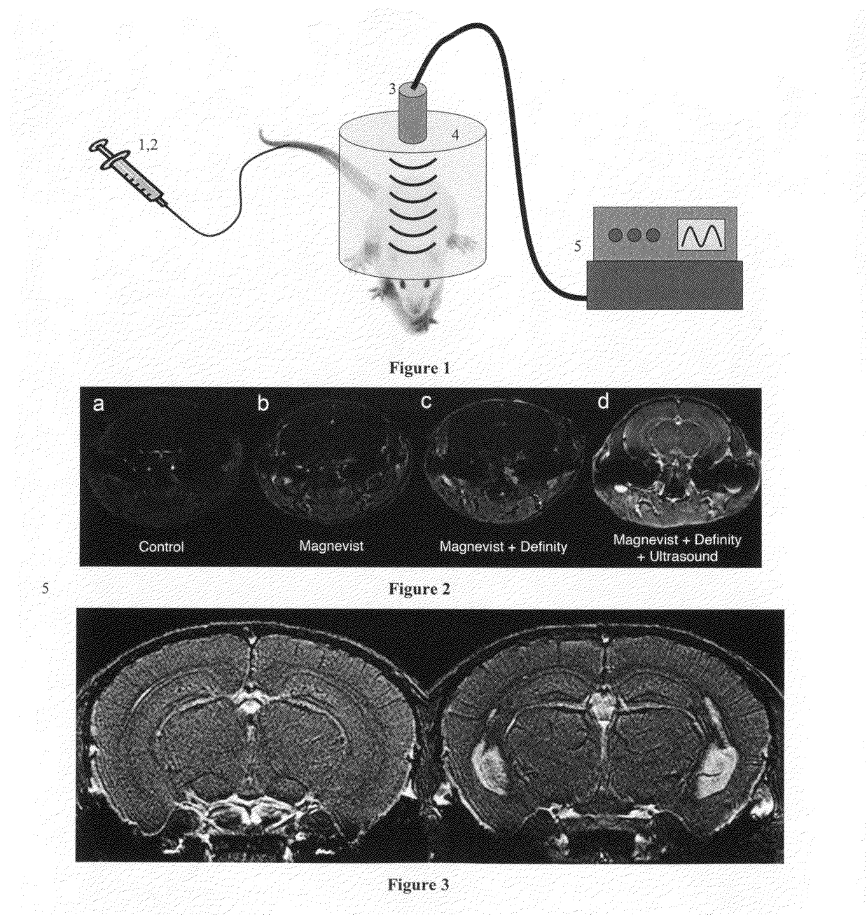

### DETAILED DESCRIPTION OF THE INVENTION

The blood-brain barrier (BBB) consists of numerous specialized features of the brain's vasculature that physically and physiologically restrict the passage of substances into the brain parenchyma. While the BBB serves a variety of important physiological functions, it also prevents the passage of most diagnostic and therapeutic agents (Muldoon L L, Soussain C, Jahnke K, Johanson C, Siegal T, Smith Q R, Hall W A, Hynynen K, Senter P D, Peereboom D M, Neuwelt E A. Chemotherapy delivery issues in central nervous system malignancy: A reality check. Journal of Clinical Oncology 2007; 25(16):2295-2305; Doolittle N D, Peereboom D M, Christoforidis G A, Hall W A, Palmieri D, Brock P R, Campbell K, Dickey D T, Muldoon L L, O'Neill B P, Peterson D R, Pollock B, Soussain C, Smith Q, Tyson R M, Neuwelt E A. Delivery of chemotherapy and antibodies across the blood-brain barrier and the role of chemoprotection, in primary and metastatic brain tumors: report of the eleventh annual blood-brain barrier consortium meeting. Journal of Neuro-Oncology 2007; 81(1):81-91; Kroll R A, Neuwelt E A. Outwitting the blood-brain barrier for therapeutic purposes: Osmotic opening and other means. Neurosurgery 1998; 42(5):1083-1099).

This invention addresses opening of the blood brain barrier using ultrasound and preformed microbubbles. It encompasses two different methods of BBB opening: (1) global opening of the BBB using unfocused ultrasound and (2) focused opening of the BBB using electronically focused (phased array) ultrasound.

Background for Global Opening with an Unfocused Transducer:

In the study of mouse models of neurological diseases, magnetic resonance microscopy (MRM) holds the promise of high-resolution, high-throughput, and longitudinal images of the mouse brain. However, the long T1 of the brain at high field has been a significant barrier. This problem has been addressed for fixed ex vivo specimens by “active staining” of the brain with T1-shortening contrast agents (Johnson G A, Cofer G P, Gewalt S L, Hedlund L W. Morphologic phenotyping with MR microscopy: The visible mouse. Radiology 2002; 222(3):789-793; Johnson G A, Ali-Sharief A, Badea A, Brandenburg J, Cofer G, Fubara B, Gewalt S, Hedlund L W, Upchurch L. High-throughput morphologic phenotyping of the mouse brain with magnetic resonance histology. Neuroimage 2007; 37(1):82-89). However, this approach does not translate well in vivo because contrast agents are excluded from the brain by the blood-brain barrier.

In addition to blocking the gadolinium-based T1-shortening agents typically used for anatomical imaging, the BBB also obstructs functional agents, such as manganese (Lin Y J, Koretsky A P. Manganese ion enhances T-1-weighted MRI during brain activation: An approach to direct imaging of brain function. Magnet Reson Med 1997; 38(3):378-388), and the new generation of targeted agents, such as labeled iron oxides (Larbanoix L, Burtea C, Laurent S, Van Leuven F, Toubeau G, Elst L V, Muller R N. Potential amyloid plaque-specific peptides for the diagnosis of Alzheimer's disease. Neurobiol Aging 2008). Indeed, the potential of these agents in the study of neurodegenerative diseases by MRM may be limited by our ability to administer them to the brain of the mouse. To better study mouse models of human disease with MRI, a technique is needed to open the BBB in the mouse both quickly and non-invasively.

A number of techniques have been tried to open the BBB. In the most common approach, a hypertonic sugar solution (e.g., arabinose or mannitol) is infused into the carotid artery (Rapoport S I. Osmotic opening of the blood-brain barrier: Principles, mechanism, and therapeutic applications. Cell Mol Neurobiol 2000; 20(2):217-230). This osmotic technique has been used in many mammals—from rats to humans—however, it has several drawbacks: it is (a) time consuming; (b) technically challenging; (c) not readily performed on mice; (d) limited to only the middle and anterior portions of one half of the brain; and (e) too invasive for longitudinal studies in small animals (Id.).

Other experimental methods for opening the BBB include mild hyperthermia (Lin J C, Lin M F. Microwave hyperthermia-induced blood-brain-barrier alterations. Radiat Res 1982; 89(1):77-87; Moriyama E, Salcman M, Broadwell R D. Blood-brain-barrier alteration after microwave-induced hyperthermia is purely a thermal effect. 1. Temperature and power measurements. Surg Neurol 1991; 35(3):177-182); direct intracerebral infusion (Kroll R A, Neuwelt E A. Outwitting the blood-brain barrier for therapeutic purposes: Osmotic opening and other means. Neurosurgery 1998; 42(5):1083-1099); and use of inflammatory mediators such as bradykinin (Abbott N J. Inflammatory mediators and modulation of blood-brain barrier permeability. Cell Mol Neurobiol 2000; 20(2):131-147). However, these methods are currently too invasive and technically challenging to be useful for global BBB disruption in the mouse.

Another tool for opening the BBB is ultrasound—focused ultrasound (FUS) can open the BBB without necessarily causing tissue damage (Mesiwala A H, Farrell L, Wenzel H J, Silbergeld D L, Crum L A, Winn H R, Mourad P D. High-intensity focused ultrasound selectively disrupts the blood-brain barrier in vivo. Ultrasound in Medicine and Biology 2002; 28(3):389-400; Vykhodtseva N I, Hynynen K, Damianou C. Histologic effects of high-intensity pulsed ultrasound exposure with subharmonic emission in rabbit brain in-vivo. Ultrasound Med Biol 1995; 21(7):969-979). If the ultrasound is administered in combination with microbubbles (i.e., ultrasound contrast agents), the acoustic pressure required for BBB disruption is lower and therefore, this ultrasoundmicrobubble combination can be used to reliably open the BBB without causing tissue damage (Hynynen K, McDannold N, Vykhodtseva N, Jolesz F A. Noninvasive M R imagingguided focal opening of the blood-brain barrier in rabbits. Radiology 2001; 220(3):640-646; Sheikov N, McDannold N, Vykhodtseva N, Jolesz F, Hynynen K. Cellular mechanisms of the blood-brain barrier opening induced by ultrasound in presence of microbubbles. Ultrasound in Medicine and Biology 2004; 30(7):979-989; McDannold N, Vykhodtseva N, Raymond S, Jolesz F A, Hynynen K. MRI-guided targeted blood-brain barrier disruption with focused ultrasound: Histological findings in rabbits. Ultrasound in Medicine and Biology 2005; 31(11):1527-1537; McDannold N, Vykhodtseva N, Hynynen K. Targeted disruption of the blood-brain barrier with focused ultrasound: association with cavitation activity. Physics in Medicine and Biology 2006; 51(4):793-807). However, most of this work has been performed in rabbits and has required surgical removal of a portion of the skull. Recently, transcranial ultrasound with microbubbles has been used to open the BBB to allow imaging agents into the brains of mice (Choi J J, Pernot M, Small S A, Konofagou E E. Noninvasive, transcranial and localized opening of the blood-brain barrier using focused ultrasound in mice. Ultrasound in Medicine and Biology 2007; 33(1):95-104; Hynynen K, McDannold N, Sheikov N A, Jolesz F A, Vykhodtseva N. Local and reversible blood-brain barrier disruption by noninvasive focused ultrasound at frequencies suitable for trans-skull sonications. Neuroimage 2005; 24(1):12-20; Bing K F, Howles G P, Qi Y, Palmeri M L, Nightingale K R. Blood-brain barrier (bbb) disruption using a diagnostic ultrasound scanner and Definity® in mice. Ultrasound in Medicine and Biology 2009; In Press). Such techniques have even been used to administer molecular imaging agents and therapeutics to the brain of a rat (Raymond S B, Treat L H, Dewey J D, McDannold N J, Hynynen K, Bacskai B J. Ultrasound enhanced delivery of molecular imaging and therapeutic agents in Alzheimer's disease mouse models. PLoS ONE 2008; 3(5):e2175).

While BBB disruption with focused ultrasound and microbubbles is non-invasive and transcranial, it is still technically challenging and limited to the small focal spots of the transducers (1-3 mm) (Choi J J, Pernot M, Brown T R, Small S A, Konofagou E E. Spatio-temporal analysis of molecular delivery through the blood-brain barrier using focused ultrasound. Physics in Medicine and Biology 2007; 52(18):5509-5530; Kinoshita M, McDannold N, Jolesz F A, Hynynen K. Targeted delivery of antibodies through the blood-brain barrier by MRI-guided focused ultrasound. Biochemical and Biophysical Research Communications 2006; 340(4):1085-1090). While such focal BBB disruption is useful for targeted delivery of therapeutic agents, for contrast-enhanced imaging of the brain, a global BBB disruption is needed. Furthermore, to be broadly adopted for the study of mouse models, the method needs to be high-throughput and technically accessible to those outside the ultrasound research community.

In this work, we present a technique to open the BBB using unfocused ultrasound or phased array focused ultrasound and microbubbles that is (a) simple and fast; (b) suitable for mice; (c) global (i.e., opens both hemispheres); (d) non-invasive; and (e) reversible. For simplicity, in this paper we refer to this technique of BBB Opening with Microbubbles and UltraSound as BOMUS. BOMUS is an acronym that subsumes BOLUS, for this document, the two terms are interchangable and have the same meaning. We employ the BOMUS technique to administer Gd-DTPA to the entire mouse brain. With the dramatically shortened T1, we are able to acquire high-resolution (50×50×100 μm) images in vivo in less than 1 hour.

Our invention involves a method that allows one to highlight (stain) specific areas in the brain of the rodent (and potentially human) to image morphology and/or function. The method employs a unique combination of the following:

a) A microbubble agent used for contrast enhancement in ultrasound; for example, Definity perflutren lipid microspheres;
b) An MRI contrast agent containing paramagnetic (or superparamagnetic) species to enhance the signal from the stained area in a magnetic resonance image; this agent may have specificity for certain tissue types; For example, MnCl₂, which causes general enhancement as well as specific enhancement of active neurons;
c) An ultrasound transducer driven by appropriate hardware; For example, a 13 mm unfocused single element immersion transducer driven by a signal generator and 25 W power amplifier;
d) Physical apparatus for coupling the transducer to the animal (or patient); for example a column of water contained by a thin plastic membrane;
e) A sequence of events including (i) intravenous (IV) injection of the microbubbles; (ii) intraperitoneal (IP) or IV injection of the magnetic resonance contrast agent; (iii) insonification of the animal (or patient) with a non destructive, series of low-pressure ultrasound pulses which interact with the microbubbles in such a fashion as to cause an opening in the blood brain barrier (BBB) in the animal (or patient).

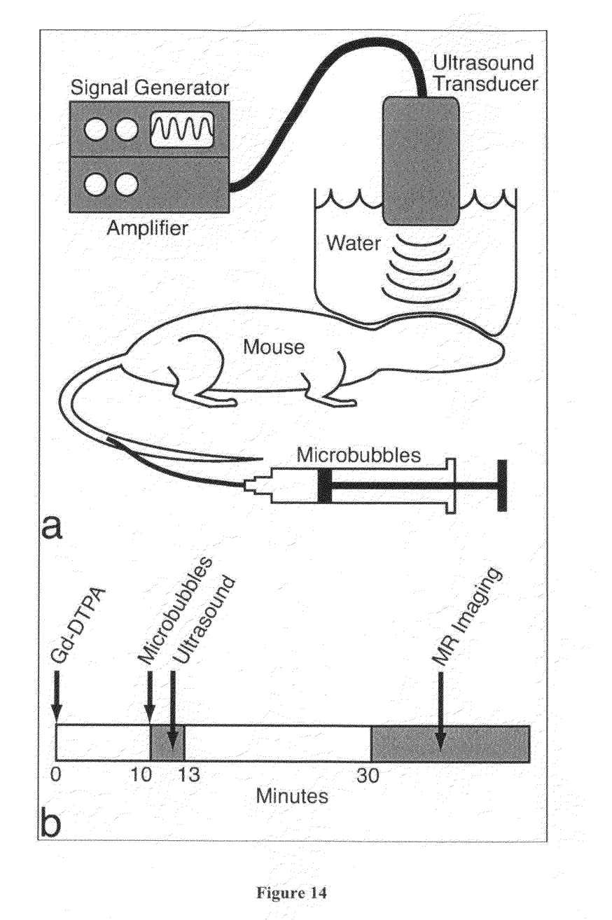

Execution of the protocol results in nondestructive opening of the blood brain barrier, transport of the MRI contrast agent across the blood-brain barrier and substantial increase in the signal from the (brain) tissues into which the MRI contrast agent has penetrated. FIG. 14 shows the physical setup used to enhance the signal in a mouse.

Background for Focused Opening with a Phased Array Transducer:

Focal opening of the BBB has great potential utility. For example, focal opening of a specific region of BBB encompassing a tumor could allow the targeted administration of toxic chemotherapeutics to the diseased region of the brain. As mentioned above, localized BBB disruption has been performed using mechanically focused ultrasound transducers in conjunction with microbubble contrast agent (such as Optison or Definity) (McDannold N, Vykhodtseva N, Hynynen K. Use of ultrasound pulses combined with Definity for targeted blood-brain barrier disruption: A feasibility study. Ultrasound Med Biol 2007; 33:584-590. McDannold N, Vykhodtseva N, Hynynen K. Blood-brain barrier disruption induced by focused ultrasound and circulating preformed microbubbles appears to be characterized by the mechanical index. Ultrasound Med Biol 2008a; 34:834-840.). This has been shown to open the BBB to allow molecules, such as gadolinium for MR contrast, imaging fluorophores for molecular imaging, and immunotherapeutics for Alzheimer's disease, to enter the brain of mice and rabbits (Hynynen K, McDannold N, Vykhodtseva N, Jolesz F. Noninvasive MR imaging-guided focal opening of the blood-brain barrier in rabbits. Radiology 2001; 220:640-646. Choi J, Pernot M, Small S, Konofagou E. Noninvasive, transcranial and localized opening of the blood-brain barrier using focused ultrasound in mice. Ultrasound Med Biol 2007; 33:95-104. Raymond S, Treat L, Dewey J, McDannold N, Hynynen K, Bacskai B. Ultrasound enhanced delivery of molecular imaging and therapeutic agents in Alzheimer's disease mouse models. PLoS One 2008; 3:1-7.). However, if a diagnostic transducer could be used for BBB disruption, it would have the advantage of providing both image guidance of the brain and therapeutic ultrasound delivery (automatically co-registered) without the need for additional devices. Furthermore, diagnostic scanners are more readily available to clinicians and researchers.

In this invention we use a diagnostic ultrasound scanner with an electronically focused ultrasound (from a phased array transducer) for locally increasing BBB permeability. The method employs a unique combination of the following:

a) A microbubble agent used for contrast enhancement in ultrasound; for example, Definity perflutren lipid microspheres;
c) A diagnostic ultrasound transducer driven by an appropriate diagnostic ultrasound scanner; For example, A Siemens Sonoline Antares diagnostic scanner and VF10-5 transducer (Siemens Medical Solutions USA, Inc., Issaquah, W A, USA);
d) Physical apparatus for coupling the transducer to the animal (or patient); for example a column of water contained by a thin plastic membrane;
e) A sequence of events including (i) intravenous (IV) injection of the microbubbles; (ii) insonification of the animal (or patient) with a non destructive, series of low-pressure ultrasound' pulses which interact with the microbubbles in such a fashion as to cause an opening in the blood brain barrier (BBB) in the animal (or patient).

### Advantages of the Invention:

1) Blood-brain barrier disruption using unfocused ultrasound: Previous work has been with mechanically focused ultrasound transducers which only allow single spots of disruption. Using an unfocused transducer allows the opening of much larger areas of the blood-brain barrier, including the whole brain.
2) Global administration of agents: previous work was interested in focal administration of agents (e.g., chemo directly to a cancer). Because we are the first to perform whole brain (or large region) blood-brain barrier disruption we can now introduce the idea of global administration of agents to the brain. We have done this with anatomical contrast agents (gadopentetate dimeglumine), functional contrast agents (manganese), and plan on doing this with targeted molecular imaging agents (e.g., iron nanoparticle-labeled nucleotides).
3) Blood-brain barrier disruption with a diagnostic phased array scanner: We also are introducing the idea of using a standard, readily available clinical scanner to perform focal blood-brain barrier disruption. There are several benefits for performing focal blood-brain barrier disruption using a phased array scanner vs. the typical mechanically focused transducer:
3a) Enormous control and flexibility for changing the focal spot size, focal spot location, ultrasound frequency, ultrasound pressure, ultrasound burst sequence, etc. This could allow a single set up to perform a wide range of blood-brain barrier disruption techniques. In contrast, a mechanical system has all parameters completely fixed, and change to the protocol requires a new transducer and change of the whole system.
3b) Combined imaging and blood-brain barrier disruption. Because these diagnostic scanners have built in imaging capability, they can be used to calibrate/precisely prescribe the location of the blood-brain barrier disruption. In contrast, mechanically focused systems require elaborate calibration and secondary imaging systems (e.g., MRI) to anatomically localize the blood-brain barrier disruption.
3c) Wide availability: clinical diagnostic scanners are ubiquitous and require no special engineering skills to set up and use.

### Additional Advantages of the Present Invention:

Our invention insonifies the entire brain to achieve a global opening of the blood-brain barrier. To the best of our knowledge, no technique has been demonstrated that achieves global opening of the blood-brain barrier. While the existing osmotic technique allow for hemispheric opening of the blood-brain barrier, it is invasive while our invention is not invasive.

Utilizing this global blood-brain barrier opening, our invention rapidly administers a contrast agent to enhance brain imaging. No other technique exists for rapidly administering contrast agents to the whole brain. While some agents can be administered to the whole brain slowly (e.g., divalent manganese will diffuse slowly into the brain), there are significant scientific and practical advantages to rapid administration. While some blood-brain barrier disruption techniques (e.g., mechanically focused ultrasound) can administer contrast agents to small portions of the brain, they have not been implemented to rapidly administer contrast to the whole brain. While some other blood-brain barrier disruption techniques (e.g., osmotic opening) can administer contrast agents to a whole hemisphere of the brain, they suffer from significant technical and scientific drawbacks (detailed above).

By choosing agents that are indicators for specific biological phenomena (such as neuronal activity), this invention can be used to identify non-morphological brain features. For example this invention can administer Mn²⁺ (a contrast agent that highlights active neurons) to the whole brain, allowing the detection of neuronal activity anywhere in the brain. No other technique of which we are aware can do this. This invention could administer any number of agents which have specificity for tissues of a certain type or nature.

Obviously, this invention could be used to administer other diagnostic and therapeutic agents, not just contrast agents. Our work to date has focused on the use for administering agents to enhance both sensitivity and specificity in imaging studies. But there is enormous potential in the use of this method to introduce and monitor the administration of a wide range of molecules for both diagnostic and therapeutic purposes.

### Embodiments of the Invention

The most immediate application of the method is the production of in vivo microscopic images of the rodent brain. Rodents (rat, mouse, gerbil, etc.) are commonly used for a wide range of basic studies. The success of MRI in the clinical arena has resulted in the production of MRI systems for basic research in small animals. Target applications for MRI of small animal are exceptionally wide ranging. But the reduction in size from man (about 70 kg) to mouse (about 25 grams) results in a commensurate increase (70,000/25=2800×) in resolution. But the signals from voxels that are 2800× smaller are 2800 weaker. Thus spatial resolution for small animal in vivo studies is usually limited by the very weak signal.

FIG. 2 demonstrates graphically how this invention can radically improve the signal in the brain of a mouse. Magnetic resonance images were acquired of a live mouse using a 7.0 T magnetic resonance system developed for magnetic resonance microscopy. A standard T1 weighted control image of the mouse with no agent or treatment (FIG. 2a) shows a dark brain. IV injection of gadopentetate dimeglumine a standard magnetic resonance contrast agent results in enhancement of the muscles (FIG. 2b) but the brain remains dark. Injection of the combination of the ultrasound contrast agent (Definity) and the magnetic resonance agent (gadopentetate dimeglumine) (FIG. 2c) again shows enhancement of the muscle but no enhancement in the brain. However, application of the ultrasound energy with the combination of Definity and gadopentetate dimeglumine opens the blood-brain barrier allowing the gadopentetate dimeglumine to actively stain the brain. Note in FIG. 2d the signal from the actively stained brain is nearly 5× (i.e., 5 fold or 5 times) stronger than it is in the unstained brain in FIG. 2a.

The enormous (nearly 5×) gain in signal can be exploited to provide enhanced spatial resolution. FIG. 3 shows 2 levels from a 3D scan of a live, actively stained mouse scanned with a relatively short (40 min) 3D scan producing 100 micron slices with 50×50 micron in plane spatial resolution. We believe these to be the highest resolution images ever obtained in a live mouse brain.

Focused ultrasound refers to the use of ultrasound to affect (e.g., open the BBB) only one area of the brain and not the complete brain. One example of focused ultrasound is the use of phased array transducer. Using any of the methods of this invention disclosed, focused ultrasound, such as focused ultrasound as produced by a phased array transducer, can transmit energy sufficient to open the BBB in no more than 50%, no more than 40%, no more than 30%, no more than 20% or no more than 10% of the complete brain.

### Use of Diagnostic Focused Ultrasound for Focal Opening:

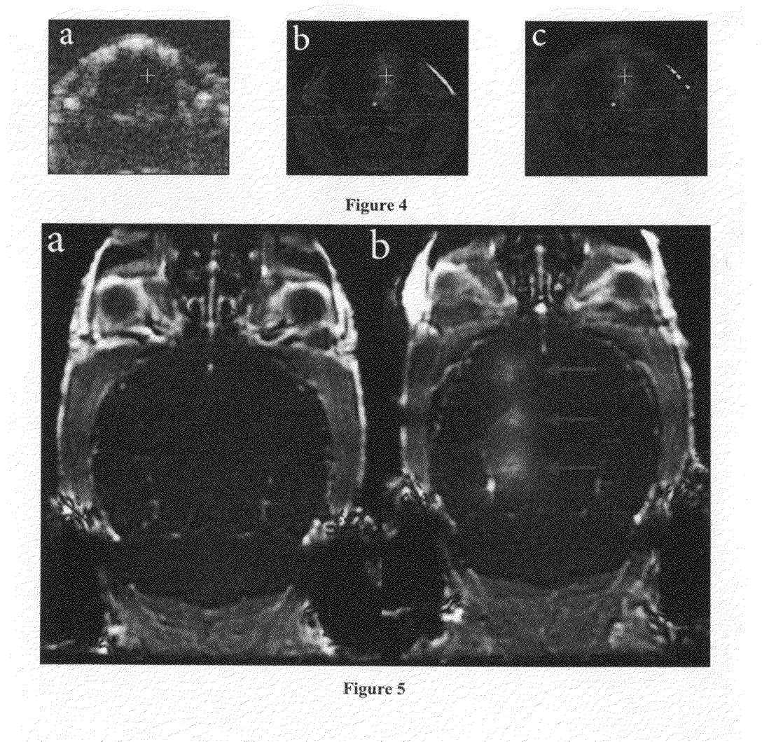

One of the novel claims is that we can use low (diagnostic) levels of ultrasound, which in turn allows us to use diagnostic ultrasound imaging systems which provide ultrasound images from which we can determine a specific region of interest in which we might selectively open the blood-brain barrier. FIG. 4 demonstrates our ability to do so. In FIG. 4 The yellow + shows the intended center of the ultrasound focus based on the B-mode image. The white region surrounding the + on the right side of the magnetic resonance image is indicative of T1 enhancement from gadopentetate dimeglumine crossing the blood-brain barrier. Blood-brain barrier opening was achieved using 5.7-MHz, 20-ms ultrasound pulses repeated at 10 Hz with an F/1.5 configuration, yielding pressures of 6.16 MPaₚₚ, in a 30-second insonification immediately after a 30-μL Definity injection.

Phased array imaging systems are known and described, for example, in U.S. Pat. Nos. 6,135,971, 6,929,608, 4,852,577, 4,670,683 and 4,414,482.

### Optimization of BOMUS

A number of variables have been explored to optimize the degree to which the BBB is opened. More specifically we have explored:

1. the relationship between does of the ultrasound agent (Definity) and the degree to which the blood-brain barrier is opened;
2. the effect of frequency of the transducer on the opening of the blood-brain barrier;
3. the effect of ultrasonic pressure on the opening of the blood-brain barrier
4. the effect of the pulse duration on the opening of the BBB.

We have explored the impact of the dose of Definity on our ability to open the blood-brain barrier by using focused ultrasound to open the blood-brain barrier in small, focal regions. Our metric is the contrast to noise ratio (CNR) i.e. the contrast between areas of the brain in which the blood-brain barrier is open and areas in which the blood-brain barrier remains in tact divided by the noise in the magnetic resonance images. FIG. 5 shows magnetic resonance images of a mouse prior to and following the application of BOMUS using a diagnostic focused, ultrasound probe. Note that 3 separate areas in which the blood-brain barrier has been opened enabling active staining of the brain surrounding the focal areas indicating the efficacy of BOMUS. In subsequent graphs, the contrast to noise ratio (CNR) is used to define the efficacy of the opening.

### Impact of Definity Dose:

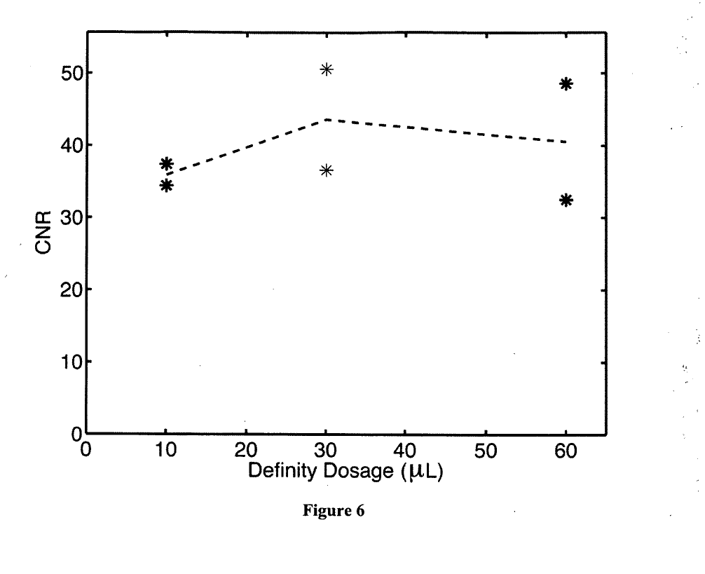

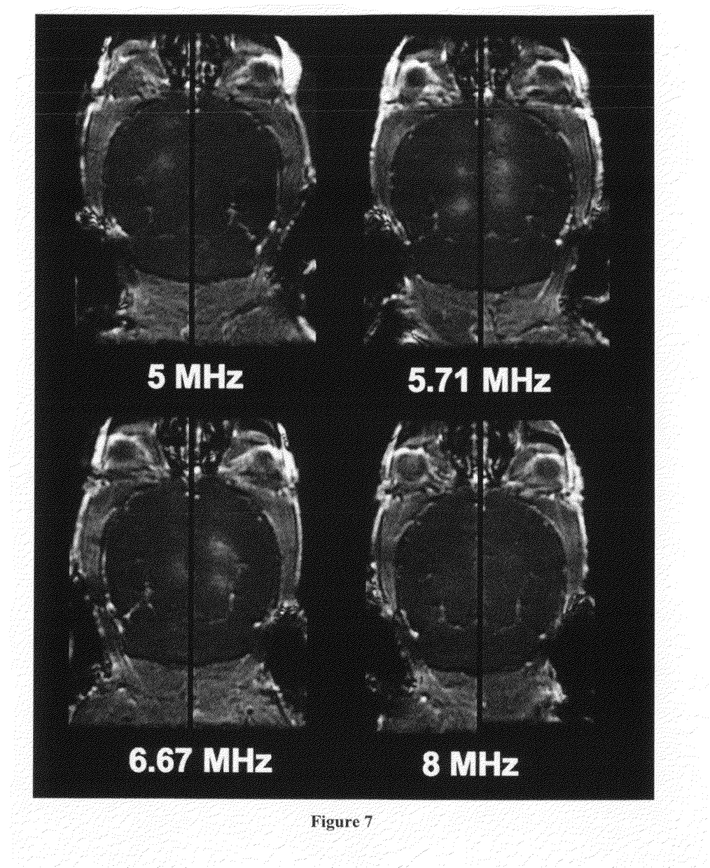

Several different doses of Definity were used to determine if there might be a dose dependency on the efficacy of the BOMUS phenomenon. The results are shown in FIG. 6 which depicts blood-brain barrier opening as a function of Definity dose. 5.7-MHz, 20-ms ultrasound pulses repeated at 10 Hz with an F/1.5 configuration, yielding pressures of 6.16 MPaₚₚ, were transmitted for 30 seconds immediately after Definity injection. Each * represents one animal and the dashed line connects the mean at each dose. The graph in FIG. 6 shows no major dose dependency.

### Impact of Ultrasound Transducer Frequency and Pressure:

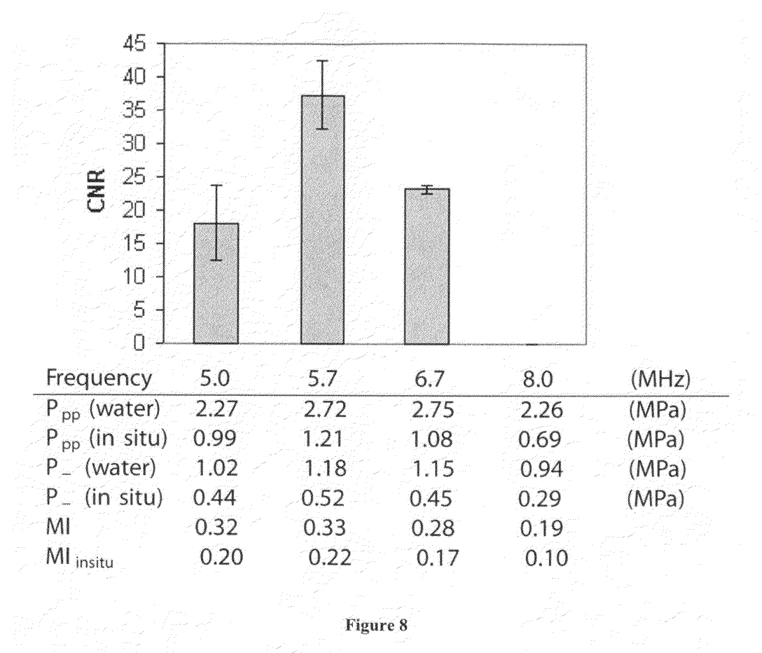

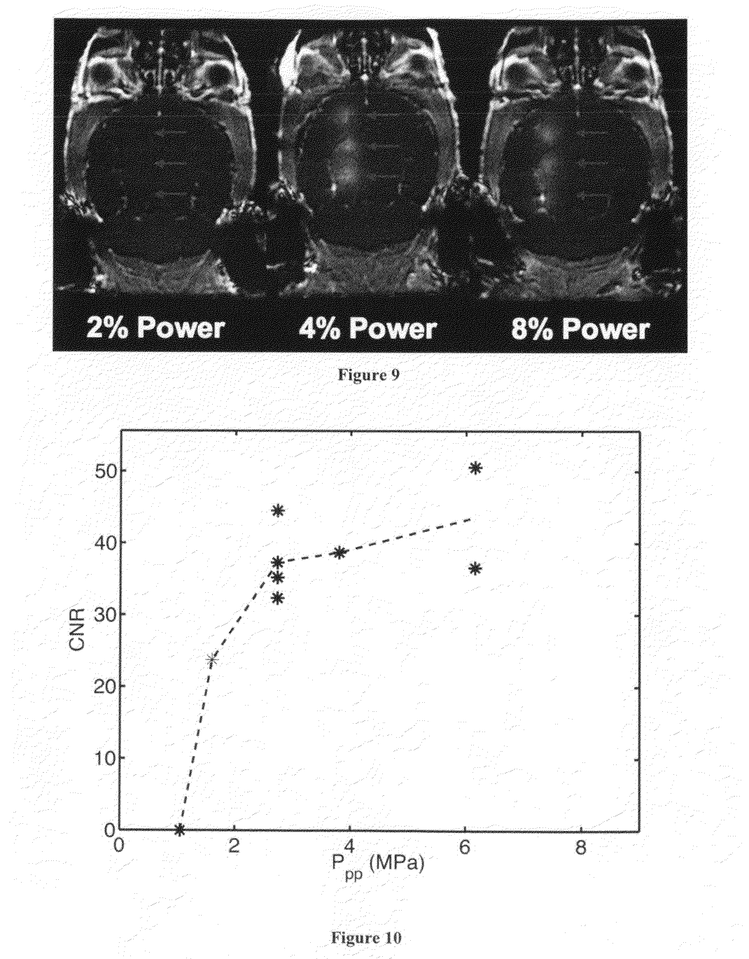

Since the blood-brain barrier is only opened when there is a combination of the ultrasound contrast agent and the application of the ultrasound energy, the mechanism that opens the blood-brain barrier must involve an interaction of the two. We hypothesize that the interaction could be frequency sensitive. Thus we have performed experiments to explore the impact of the frequency of the ultrasound transducer on the efficacy of opening the blood-brain barrier. Again the metric we use to measure the impact of the frequency is the contrast to noise ratio (CNR). FIG. 7 shows the magnetic resonance images of live mice imaged with focused ultrasound at 4 different frequencies. Each experiment was conducted on two mice focusing on either the left or right hemisphere. The composite shows the variability between specimens. Note that the effect is considerably greater at 5.71 MHz and nonexistent at 8 MHz. FIG. 8 shows blood-brain barrier opening for ultrasonic frequencies from 5 to 8 MHz for the same input voltage. 20-ms ultrasound pulses repeated at 10 Hz with an F/1.5 focal configuration were transmitted for 30 seconds immediately after a 30-μL Definity injection. At least two animals were tested per frequency. The standard deviation of these pressure measurements are ≦1%. In this figure, Ppp refers to Peak to peak pressure, P− refers to peak negative pressure, MI is mechanical index, and MPa is a unit of pressure: megapascals. FIG. 9 shows magnetic resonance images of live mice receiving BOMUS intervention at varied power levels. Note that below 2% of the peak power there is no opening of the blood-brain barrier. Given the configuration of the system, the percentage of peak power corresponds directly to the ultrasonic pressure. The quantitative analysis of this experiment is shown in FIG. 10. In FIG. 10, the effect of ultrasonic pressures (peak to peak) from 1.05 to 6.16 MPa (non-derated) on blood-brain barrier opening. The experiment was performed at 5.7-MHz, 20-ms ultrasound pulses repeated at 10 Hz with an F/1.5 configuration were transmitted for 30 seconds immediately after a 30-μL Definity injection. Each * represents one animal and the dashed line connects the mean at each pressure.

### Impact of Pulse Duration and Number of Cycles:

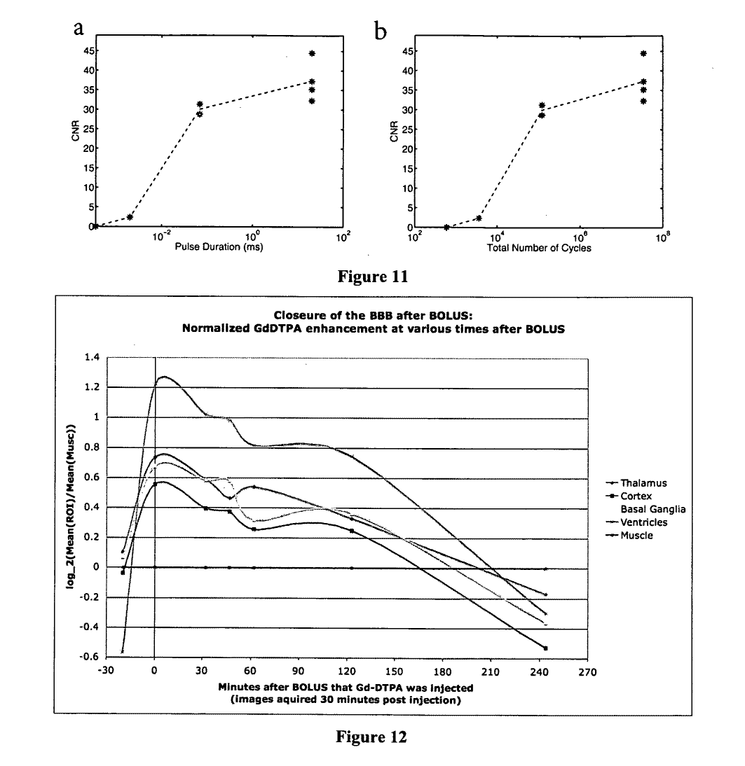

There are a wide range of parameters at our disposal in optimizing the opening of the blood-brain barrier. The fact that many of these parameters show threshold effects, and that all of these effects are seen at energy levels far below the energies required for heating, attest to the fact that the method involves a mechanism beyond that of simple heating as has been exploited in previous use of ultrasound to open the blood-brain barrier. Again we use contrast to noise ratio (CNR) in the magnetic resonance images acquired after disruption of the blood-brain barrier as a metric of efficacy. Our results are shown in FIG. 11. FIG. 11a depicts the effect of pulse durations of 0.35 microsecond (B-mode), 2 microsecond (Color Doppler), 70 microsecond (Acoustic Radiation Force Impulse Imaging), and 20 millisecond on blood-brain barrier opening. 5.7-MHz ultrasound pulses repeated at 10 Hz with an F/1.5 (Ultrasound aperture) configuration yielding 2.72 MPa were transmitted for 30 seconds immediately after a 30-μL Definity injection. Each * represents one animal and the dashed line connects the mean at each pulse duration. FIG. 11b depicts the same data presented as a function of the total number of cycles in the insonification sequence. Note these are semi-log plots in x. In this figure, MPaₚₚ is peak to peak pressure measured in MPa. Definity and micro bubble are given IV. The most convenient location in the mouse being the tail vein.

### Length of Opening

We have performed animal studies to determine the time during which the blood-brain barrier is open and any potential biological hazard to the process. Experiments were performed on anesthetized animals in which the wide area transducer was used to open the blood-brain barrier in the entire brain. For these studies, the efficacy of the method to open the blood-brain barrier was assessed by looking at the signal in a region of interest in specific regions of the brain normalized to the signal in the muscle in the jaw. Our results are shown in FIG. 12. In FIG. 12, GdDTPA is Gadopentetate Dimeglumine, which is the generic name for Magenvist (Bayer HealthCare Pharmaceuticals Inc. Wayne, N.J. 07470), Mean(ROI) is mean over the region of interest (i.e., thalamus, cortex, etc) and Mean(Musc) is the mean over the muscle region. Since muscle does not have a BBB we use it to normalize the brain data.

### Histology

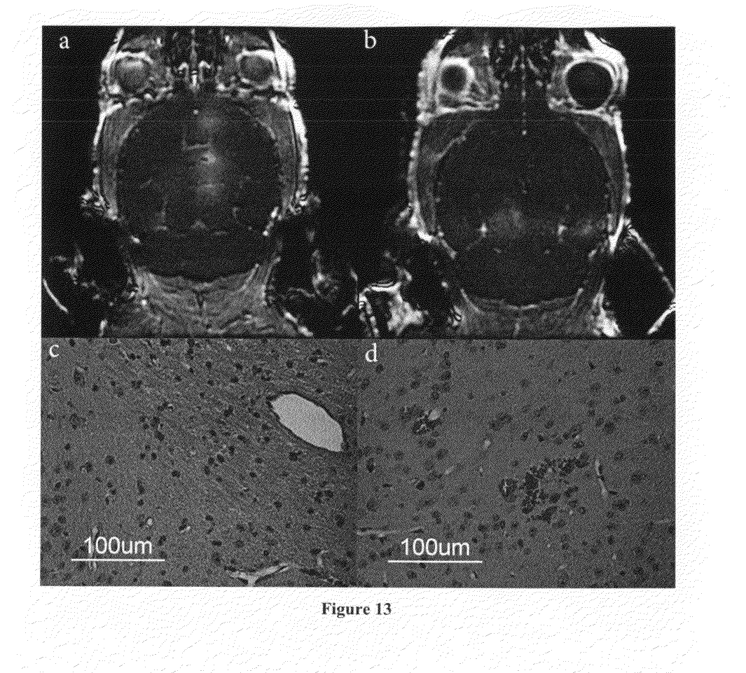

Histology is the most frequent gold standard for determining the safety of a procedure or process. FIG. 13 shows magnetic resonance images (top) of a) animal exposed to ultrasound at power levels used for BOMUS and b) at higher power levels. Histology (bottom) of c) the animal submitted to BOMUS shows no tissue damage while d) the histology from the animal exposed to higher levels shows clear evidence of hemorrhage.

Our invention offers significant improvements over current methods. Previous works have referred to the use of mechanically focused ultrasound transducers. In contrast, the work in this disclosure is made with unfocused transducers and diagnostic phased-array (electronically focused) transducers. This difference directly contributes to at least four benefits compared to current methods.

1. Blood-brain barrier disruption (BBBD) using unfocused ultrasound transducers: Previous work has been with mechanically focused ultrasound transducers which only allow single spots of disruption. Using an unfocused transducer allows the opening of much larger areas of the BBB (including the whole brain of small animals). Our unfocused transducer has only a single element, but the work could be done with multiple unfocused transducers the unfocused transducers require only a signal generator and power amplifier to run, the produce no images. As shown in this disclosure and our experiments, multiple unfocused transducers, such as in a diagnostic phrased array, may be used to practice the methods of this invention.

2. Global administration of agents to animals (e.g., from large animals to small animals including large and small mammals): previous work was interested in focal administration of agents (e.g., chemo directly to a cancer). Because we are the first to perform whole brain (or large region) blood-brain barrier disruption we can now introduce the idea of global administration of agents to the brain. Such agents can be diagnostic (e.g., contrast agents for imaging) or therapeutic (e.g., chemotherapy). We have done this with anatomical contrast agents (magnevist), functional contrast agents (manganese), and plan on doing this with targeted molecular imaging agents (e.g., iron nanoparticle-labeled nucleotides).

3. Focal blood-brain barrier disruption with a phased array ultrasound system: We also are introducing the idea of using a standard, readily available clinical scanner to perform focal blood-brain barrier disruption. There are several benefits for performing focal blood-brain barrier disruption using a phased array scanner vs. the typical mechanically focused transducer:

The advantages include enormous control and flexibility for changing the focal spot size, focal spot location, ultrasound frequency, ultrasound pressure, ultrasound burst sequence, etc. This could allow a single set up to perform a wide range of blood-brain barrier disruption techniques. In contrast, a mechanical system has all parameters completely fixed, and change to the protocol requires a new transducer and change of the whole system.

Combined imaging and blood-brain barrier disruption. Because these diagnostic scanners have built in imaging capability, they can be used to calibrate/precisely prescribe the location of the blood-brain barrier disruption. In contrast, mechanically focused systems require elaborate calibration and secondary imaging systems (e.g., MRI) to anatomically localize the blood-brain barrier disruption.

Wide availability: clinical diagnostic scanners are ubiquitous and require no special engineering skills to set up and use.

4. We also discovered the idea of using the co-administration of imaging contrast agent and therapeutic to monitor the administration of the therapeutic agent. For example opening the BBB to administer chemotherapy to a brain tumor, but having an imaging agent mixed in with the chemotherapy to that the exact extent of the administration can be evaluated or monitored in real time.

Suppliers: The Supplier for Various Agents and Chemicals Used in this Disclosure and/or Their Tradenames, are Listed Below:
Optison, (Octafluoropropane/Albumin) GE Healthcare Inc., Princeton, N.J. 08540). Perflutren lipid microsphere (Definity, Lantheus Medical Imaging, North Billerica, Mass.). Galactose-Palmitic Acid microbubble suspension (Levovist, Bayer HealthCare Pharmaceuticals Inc. Wayne, N.J.). Air/Albumin (Albunex, Mallinckrodt, Inc. Hazelwood, Mo. and Quantison, Quandrant, Notingham, UK). Air/Palmitic acid (Levovist/SHU508A, Schering AG, Berlin, Germany). Perfluoropropane/Phospholipids (MRX115, DMP115). Dodecafluoropentane/Surfactant (Echogen/QW3600, Sonus Pharmaceuticals, Bothell, W A). Perfluorobutane/Albumin (Perfluorocarbon exposed sonicated dextrose albumin). Perfluorocarbon/Surfactant (QW7437). Perfluorohexane/Surfactant (Imagent/AFO150 Alliance Pharmaceutical Corp. San Diego, Calif.). Sulphur hexafluoride/Phospholipids (Sonovue/BR1 Bracco Diagnostics Inc. Princeton, N.J.). Perfluorobutane/Phospholipids (BR14, Bracco Diagnostics Inc. Princeton, N.J.). Air/Cyanoacrylate (Sonavist/SHU563A, Schering AG, Berlin, GERMANY). Perfluorocarbon/Surfactant (Sonazoid/NC100100, GE Healthcare Inc., Princeton, N.J. 08540). Magnetic resonance agents: gadopentetate dimeglumine (Magnevist, Bayer HealthCare Pharmaceuticals Inc. Wayne, N.J.). Gadodiamide (Omniscan, GE Healthcare, Princeton, N.J.). Gadoteridol (ProHance, Bracco Diagnostics Inc. Princeton, N.J.). Gadobenate dimeglumine (MultiHance, Bracco Diagnostics Inc. Princeton, N.J.), or gadoversetamide. X-ray contrast agents: iopromide (Ultravist, Bayer HealthCare, Wayne, N.J. 07470). Iopamidol (Isovue, Bracco Diagnostics Inc. Princeton, N.J.). Ioversol, or Iodixanol (Visipaque, GE Healthcare, Princeton, N.J.), lobitridol.

### References (not Necessarily Prior Art):

1. Johnson G A, Cofer G P, Gewalt S L, Hedlund L W. Morphologic phenotyping with MR microscopy: the visible mouse. Radiology 2002; 222(3):789-793. 2. Kroll R A, Neuwelt E A. Outwitting the blood-brain barrier for therapeutic purposes: Osmotic opening and other means. Neurosurgery 1998; 42(5):1083-1099. 3. Deng S X, Panahian N, James H, Gelbard H A, Federoff H J, Dewhurst S, Epstein L G. Luciferase: a sensitive and quantitative probe for blood-brain barrier disruption. J Neurosci Methods 1998; 83(2):159-164. 4. Hynynen K, McDannold N, Vykhodtseva N, Jolesz F A. Noninvasive MR imaging-guided focal opening of the blood-brain barrier in rabbits. Radiology 2001; 220(3):640-646. 5. Mesiwala A H, Farrell L, Wenzel H J, Silbergeld D L, Crum L A, Winn H R, Mourad P D. High-intensity focused ultrasound selectively disrupts the blood-brain barrier in vivo. Ultrasound Med Biol 2002; 28(3):389-400. 6. Choi J J, Pernot M, Small S A, Konofagou E E. Noninvasive, transcranial and localized opening of the blood-brain barrier using focused ultrasound in mice. Ultrasound Med Biol 2007; 33(1):95-104. 7. McDannold N, Vykhodtseva N, Hynynen K. Use of ultrasound pulses combined with definity for targeted blood-brain barrier disruption: A feasibility study. Ultrasound Med Biol 2007; 33(4):584-590. 8. McDannold N, Vykhodtseva N, Hynynen K. Targeted disruption of the blood-brain barrier with focused ultrasound: association with cavitation activity. Phys Med Biol 2006; 51(4):793-807. 9. Sheikov N, McDannold N, Vykhodtseva N, Jolesz F, Hynynen K. Cellular mechanisms of the blood-brain barrier opening induced by ultrasound in presence of microbubbles. Ultrasound Med Biol 2004; 30(7):979-989. 10. Journal Of Ultrasound In Medicine 28 (7): 871-880 July 2009. 11. U.S. Pat. No. 5,752,515. 12. U.S. Pat. No. 6,716,168. 13. U.S. application Ser. No. 11/370,094 (Publication Number 20060241529). 13. Frinkley K, Howles-Banerji G P, Qi Y, Johnson G A, Nightingale K R. Blood-Brain Barrier Disruption Using a Diagnostic Scanner and Definity in Mice. J Acoust Soc Am. 2008; 123(5):3218.

### EXAMPLES

### Example 1

### In Vivo Magnetic Resonance Microscopy by BOMUS

### Methods

### Microbubbles

Prior to opening the BBB, perflutren lipid microspheres (Definity, Lantheus, N. Billerica, Mass.) were produced by “activating” the vial (i.e., shaking it in the manufacturer-supplied device for 45 seconds) according to the prescribing information sheet. Immediately prior to microbubble administration, the vial was agitated by hand for 1 minute.

### Ultrasound System

For insonification a circular single-element ultrasound transducer (model A382S-SU, Olympus NDT) was used, which had a diameter of 13 mm and a center frequency of 2.15 MHz. See, FIG. 14 where panel a depicts the BOMUS setup and panel b depicts the experimental timeline. The transducer was positioned using a 3-axis frame (VisualSonics, Toronto, ON) at its natural focal distance (58 mm) in the water column directly over the mouse brain. The natural focus distance (i.e., the Rayleigh distance) was estimated as d2/4 lambda, where d is the element diameter and lambda is the wavelength in water (Ultrasonic Transducers Technical Notes. Technical brochure: Olympus NDT, Waltham, Mass.; March 2006. 11 p). The transducer was driven by a 50 dB power amplifier (model 240L, E&I, Rochester, N.Y.), which was connected to a signal generator (model 33220A, Agilent, Santa Clara, Calif.) that produced the 3-minute ultrasound pulse sequence. Two pulse sequences were used with different acoustic pressures, but equivalent average power output. The pulse amplitude (mVpp) input into the power amplifier was calibrated using a hydrophone (described below) to generate peak-negative acoustic pressures of either 0.36 MPa or 0.52 MPa at the center of the transducer's natural focus. Two sinusoidal pulse sequences of different pressures were used. The lower pressure sequence parameters were amplitude=0.167 mVpp (0.36 MPa), pulse length=50000 cycles, pulse repetition frequency=15.6 Hz; and the higher pressure sequence parameters were 0.258 mVpp (0.52 MPa), 32,000 cycles, 10 Hz. These pulse sequences were selected so that each sequence applied an average power of approximately 2 W to the transducer—a power that was unlikely to damage the transducer.

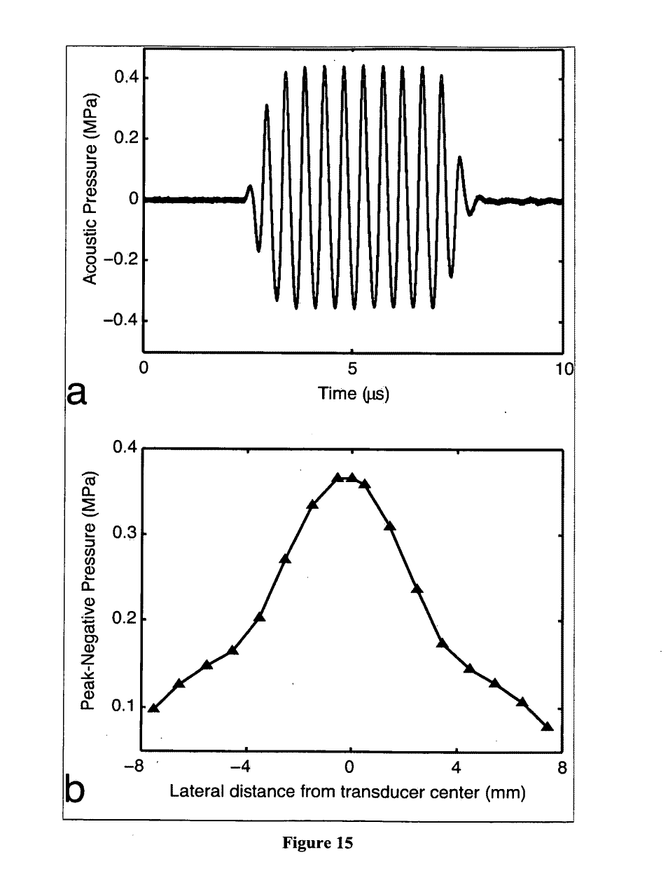

To calibrate the pulse amplitude (voltage applied to the power amplifier) with the acoustic pressure generated by the transducer, measurements were made in water using a hydrophone (model SN S4-251, Sonora, Longmont, Colo.) with a 0.4 mm spot size membrane. The calibration pulse (FIG. 15a) had a length of 10 cycles, a pulse repetition frequency of 10 Hz (PRF=10 Hz), and amplitudes ranging from 50 to 400 mVpp. An input voltage of 167 mVpp produced a peak negative pressure of 0.36 MPa. A step motorcontrolled translation stage (Newport Corporation, Irvine, Calif.) operated by a custom LabVIEW program (National Instruments, Austin, Tex.) was used to measure the lateral acoustic pressure profile at the natural focus. In FIG. 15b the lateral profile of the beam was measured at the transducers natural focus (58 mm) and the results are shown.

BBB Opening with Microbubbles and Ultrasound (BOMUS)

All animal studies were approved by the Duke University Institutional Animal Care and Use Committee. A total of 26 C57BL/6 mice were used in this study. For all procedures, mice were anesthetized with isoflurane by nose cone. The respiratory rate was maintained between 85 and 125 breaths per minute by titrating the isoflurane concentration. Body temperature was maintained using a heat lamp (during BOMUS) or blown air (during MRI). The nose cone apparatus (Howles G P, Nouls J C, Qi Y, Johnson G A. Rapid production of specialized animal handling devices using computer-aided design and solid freeform fabrication. J Magn Reson Imaging 2009; In Press) was manufactured to fix the animal's head precisely and reliably in the “skull-flat” position (i.e., the dorsal skull surface is horizontal).

Prior to ultrasound, hair was removed from the scalp of the mouse using either a trimmer or a depilatory agent (Nair®, Church & Dwight, Princeton, N.J.). Ultrasound gel was placed on the scalp, and then, a column of water contained by a 7.6-μm (0.3 mil) plastic sheet was lowered onto the head (FIG. 14a). In this water column, the ultrasound transducer was centered over the mouse brain, 58 mm above the scalp. A hemicylindrical plastic shield was placed over the thorax to prevent the water column from applying pressure to the body.

To open the BBB, 30 microliters of perflutren lipid microspheres (activated Definity) were injected through a tail vein catheter and simultaneously the ultrasound pulse sequence was initiated. The ultrasound was applied for 3 minutes.

### MR Imaging

To enhance the brain with MR contrast, Gd-DTPA (Magnevist, Bayer HealthCare Pharmaceuticals, Wayne, N.J.) was administered by intraperitoneal (IP) injection 10 minutes prior to BOMUS (FIG. 14b). The 10-minute delay was chosen because in preliminary studies, it was found that most of the enhancement from an IP injection of Gd-DTPA occurs within 10-15 minutes post-injection. The Gd-DTPA dose was 3.2, 6.4, or 9.5 mmol/kg, as noted later. After BOMUS, high-throughput MR images were acquired. Because Gd-DTPA is normally excluded by the BBB, opening of the BBB was indicated by contrast-enhancement on T1-weighted MRI.

For MRI, a 35 mm diameter quadrature transmit/receive volume coil (m2m Imaging Corp., Cleveland, Ohio) was used. The MR system was a 7 T horizontal bore magnet driven by a GE EXCITE console (General Electric Healthcare, Milwaukee, Wis.). MR images were acquired using either a high-throughput or high-resolution protocol. The high-throughput scan (3.2 minutes) used a 3D spoiled gradient recalled (SPGR) sequence with the following parameters: repetition time (TR)=25 ms; echo time (TE)=2 ms; flip angle (FA)=30 degrees; field of view (FOV)=20×20×12 mm; matrix=128×128×60; number of averages (NEX)=1. Data were acquired at a resolution of 156×156×200 micrometers.

High-resolution images were acquired with a similar 51 minute SPGR protocol: TR=25 ms; TE=3-4 ms; FA=15-22 degrees; FOV=20×20×8 mm; matrix=384×384×80; NEX=4. Data were acquired at a resolution of 52×52×100 micrometers.

T1 measurements were performed by acquiring a series of 2D spin echo images with varying TRs: TR=200, 400, 800, 1600, 3200, 6400, or 12800 ms; TE=7 ms; BW=31.25; slice thickness=1 mm; FOV=20×20 mm; matrix=128×128. T1 over a region of interest (ROI) was estimated using a three-parameter non-linear fit of the data to the following equation: I=m(1−e(−T1/TR))+a, where I is the mean ROI intensity and TR is the repetition time. The three terms that were fit were m, a multiplicative constant; a, an additive constant; and T1.

### Duration of BBB Disruption

To determine the duration of the BBB opening, BBB opening was assayed at several time intervals after BOMUS. For each time interval (0, 30, 45, 60, 120, or 240 minutes), a different animal was used. The BBB was opened with BOMUS and after the specified delay, Gd-DTPA (0.167 M) was administered by tail vein (1 mmol/kg). A high throughput image was acquired 30 minutes later.

### Histology

To determine if the BOMUS procedure caused tissue damage, brain sections from selected mice were examined by light microscopy. After MR imaging, the mice were transcardially perfused first with saline (5 minutes) and then with 10% formalin (5 minutes). The fixed brains were embedded in paraffin and 4-micrometers sections were taken at 500-micrometers intervals throughout the entire brain. Hematoxylin and eosin-stained (H&E) sections were then examined for instances of red blood cell extravasation into the brain parenchyma.

### Behavioral Assessment

The effect of the BBB disruption procedure on behavior was assessed using selected components of the well-established test battery developed by Irwin in 1968 (Irwin S. Comprehensive Observational Assessment: Ia. A systematic quantitative procedure for assessing behavioral and physiologic state of mouse. Psychopharmacologia 1968; 13(3):222-257). A subset of 16 tests was selected that in our preliminary work yielded the most consistent measurements. Our protocol included the following tests, described in detail in reference (Id.): body position, locomotor activity, transfer arousal, spatial locomotion, startle, tail elevation, touch-escape, positional passivity, grip strength, body tone, toe-pinch, limb tone, abdominal tone, provoked biting, tail-pinch, and righting reflex. These tests are all scored on a scale from 0 to 8, where higher numbers correspond with a higher level of activity, arousal, and responsiveness. (Note: To be consistent with the other tests, the scale for the righting reflex was reversed from its original description in (Irwin S. Comprehensive Observational Assessment: Ia. A systematic quantitative procedure for assessing behavioral and physiologic state of mouse. Psychopharmacologia 1968; 13(3):222-257).) The scores from these individual tests were summed to calculate an overall behavior score.

The testing protocol was performed at three different time points: prior to BOMUS; approximately 3 hours after recovery from anesthesia; and approximately 24 hours after recovery from anesthesia. Because of the experimental schedule, the baseline testing was performed consistently in the early morning, shortly after the mice had been transferred from the vivarium in a fresh cage. In contrast, the 24-hour post-anesthesia testing was consistently performed in the afternoon after the mice had been in the new cage for a full day. The protocol was administered to both BOMUS-treated (n=8) and control animals (n=3). The control animals were handled identically (i.e., isoflurane anesthesia, hair removal, Gd-DTPA) except they did not receive ultrasound or microbubbles.

### Results

### Ultrasound Beam Characterization

The unfocused ultrasound beam was characterized in water using a hydrophone. The hydrophone was positioned at the center of the ultrasound beam at the transducer's natural focal distance (i.e., the Rayleigh distance), 58 mm. Applying a 10-cycle sinusoidal pulse (PRF=10 Hz), the acoustic amplitude scaled linearly (R2=0.9992) over the input range of 50 to 400 mVpp (FIG. 2 a). Input voltages of 258 and 167 mVpp corresponded to peak-negative acoustic pressures of 0.52 and 0.36 MPa. At the natural focal distance, the beam's lateral full-width-at-half-maximum was 7.4 mm (FIG. 15b).

### Opening of the BBB

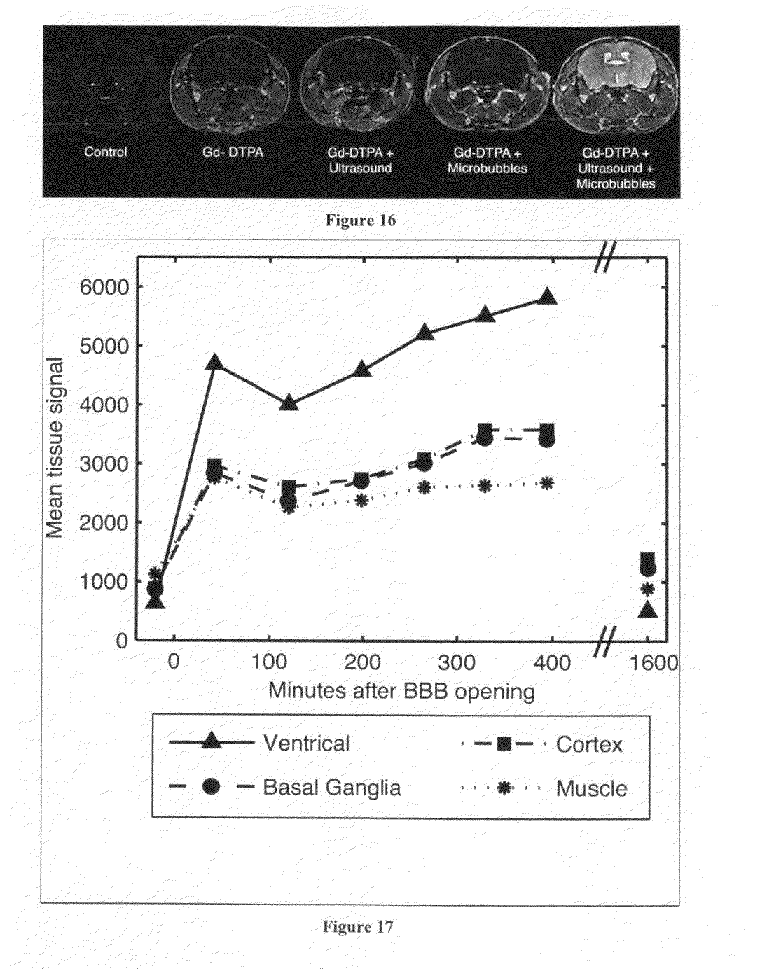

To determine if the combination of unfocused ultrasound and microbubbles could be used to globally open the BBB, this treatment was compared to a variety of control scenarios (FIG. 16). In FIG. 16, T1-weighted SPGR images (high-throughput protocol) demonstrate that Gd-DTPA enhances body tissues but is excluded from the brain by the intact BBB. Treatment with either ultrasound or microbubbles alone does not make the BBB permeable to Gd-DTPA. However, co-administration of ultrasound and microbubbles globally opens the BBB, allowing the Gd-DTPA to enhance the brain. Animals received Gd-DTPA 6.4 mmol/kg IP 10 minutes prior to treatment, and T1-weighted images (high-throughput protocol) were acquired 20 minutes after treatment (FIG. 14b).

All animals receiving Gd-DTPA had enhancement of the tissues surrounding the brain (e.g., skin, muscle, and salivary glands), as well a slight enhancement of the choroid plexus (which does not have the BBB and is relatively permeable (Segal M B. The choroid plexuses and the barriers between the blood and the cerebrospinal fluid. Cellular and Molecular Neurobiology 2000; 20(2):183-196)). Those animals receiving no treatment, only ultrasound, or only microbubbles had no enhancement in the cerebrospinal fluid (CSF) or in the brain parenchyma. However, those mice receiving both ultrasound and microbubbles simultaneously had a dramatic enhancement in the CSF and brain parenchyma.

While this dose of Gd-DTPA (6.4 mmol/kg IP) provided excellent enhancement at 30 minutes post-injection, for imaging at subsequent time points (45 minutes and beyond) 6.4 mmol/kg Gd-DTPA was excessive. As the Gd-DTPA continued to diffuse out of the peritoneal space and into the blood stream and body tissues, some tissues showed a decrease in signal (data not shown). This signal drop was presumably due to the T2-relaxivity of Gd-DTPA dominating the T1-relaxivity at higher concentrations. For this reason, subsequent experiments were conducted using 1.0 or 3.2 mmol/kg Gd-DTPA.

### Time Course of Enhancement

To examine the temporal pattern of enhancement, T1-weighted images (high throughput protocol) were acquired at three time points prior to BOMUS (n=8), serially over 6 hours after BOMUS (n=4), and 27 hours after BOMUS (n=1). Signal measurements were taken from ROIs placed in the cortex, basal ganglia, lateral ventricle, and jaw muscle (FIG. 17). (The cortex and basal ganglia were chosen in order to sample both superficial [cortex] and deep structures [basal ganglia] of the brain.) In FIG. 17 a time-course of Gd-DTPA enhancement in the brain and muscle after BOMUS. T1-weighted images (high-throughput protocol) were acquired prior to BOMUS (plotted at time<0), serially after BOMUS, and 27 hours after BOMUS. ROIs were placed in the jaw muscle, lateral ventricle, cerebral cortex, and basal ganglia to measure the mean signal intensity. Data are from a single mouse except times<0 and 27 hours, which are from separate mice. Immediately after BOMUS, all tissues show a dramatic signal enhancement. This enhancement diminished slightly over the first two hours, but then steadily increased over the next four hours. However, by the next day, the tissue signal had returned to the pre-BOMUS baseline levels.

### Duration of BBB Opening

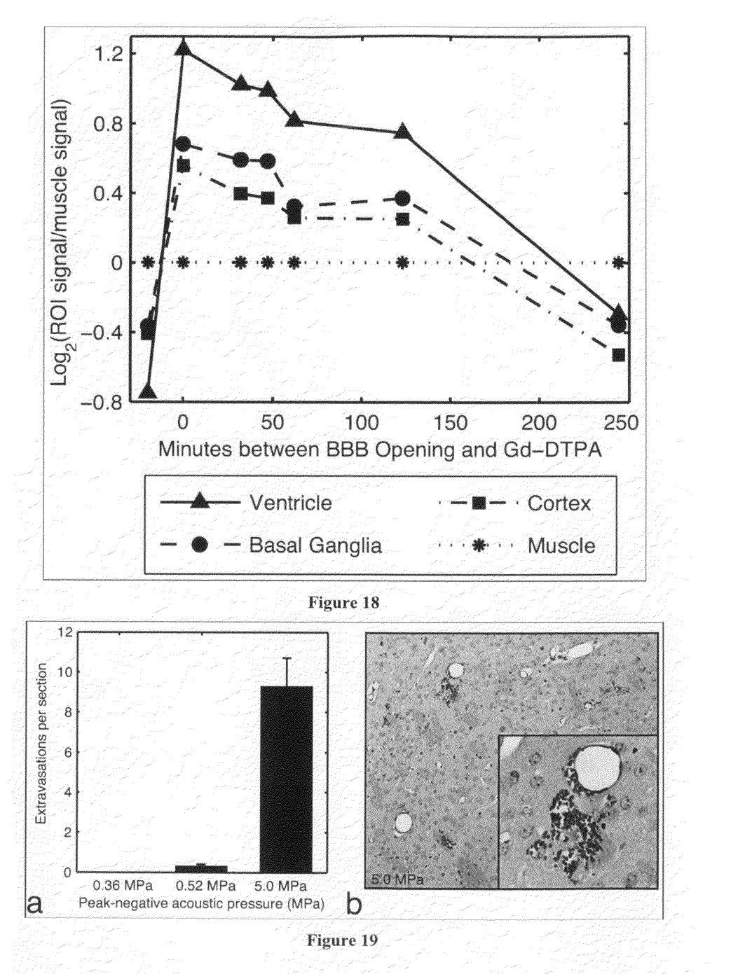

To examine the duration of BBB opening, the BBB permeability was assayed by injecting Gd-DTPA at several time intervals after BOMUS (FIG. 18). In FIG. 18, the duration of BBB disruption was demonstrated by assaying BBB permeability at several times after BOMUS. Signal measurements were made in several ROIs from T1-weighted images (high-throughput protocol). To account for inter-animal variability, the muscle signal was used to normalize the intracranial signals: log 2 (tissue signal/muscle signal) is plotted along the y-axis. For comparison, data from an untreated control animal is shown at time<0. Each time interval was assayed using a separate animal (n=7). Signal measurements were made from ROIs placed in the lateral ventricles, basal ganglia, cortex, and jaw muscle. Because the muscle was not affected by the BOMUS procedure, the muscle signal was used to normalize the values of the intracranial ROIs. These post-BOMUS animals were compared to a control animal that received IV Gd-DTPA but no BOMUS (shown at time<0 min in FIG. 18).

As assayed with Gd-DTPA, BBB permeability was greatest during the BOMUS procedure. After BOMUS, the permeability decreased steadily over the 2 hours. Between 2 and 4 hours after BOMUS, the BBB permeability dropped more quickly, such that by 4 hours, enhancement was comparable to the pre-BOMUS levels in all tissues except the ventricles, which had some slight residual enhancement.

### Histology

To determine if the BOMUS procedure caused tissue damage, the brains of selected BOMUS-treated mice (n=8) were examined with light microscopy. Sections were taken at 500 μm intervals, providing approximately 14 sections per brain. In previous reports using focused ultrasound and microbubbles, microhemorrhage (i.e., red blood cell extravasation in the brain parenchyma) was found to be a reliable early indicator of tissue damage (Hynynen K, McDannold N, Sheikov N A, Jolesz F A, Vykhodtseva N. Local and reversible blood-brain barrier disruption by noninvasive focused ultrasound at frequencies suitable for trans-skull sonications. Neuroimage 2005; 24(1):12-20). Therefore, in this study, brain sections from selected animals were examined for extravasations and the number of extravasations seen on each slide was tallied (FIG. 19a). In FIG. 19a, the mean number of red blood cell extravasations seen in each histology slide of the brain is shown for acoustic pressures of 0.36 MPa (n=3), 0.52 MPa (n=4), and 5.0 MPa (n=1). Error bars show standard error. Two global BOMUS treatment groups were examined: peak-negative acoustic pressure of 0.52 MPa (n=4) and 0.36 MPa (n=3). For comparison, a brain was examined from a mouse that underwent BOMUS using a B-mode scan from a commercial ultrasound system (peak-negative pressure=5.0 MPa). Note that while the global BOMUS groups had ultrasound applied to the whole brain, the B-mode BOMUS only insonified in a 2 mm axial slab—approximately ⅙th of the brain volume. To account for variations in the number of sections prepared from each brain, the data is reported in “extravasations per section.”

The brains of animals treated with 0.36 MPa BOMUS had no identifiable extravasations. The brains of animals treated with 0.52 MPa showed only 0.3 extravasations per section. Interestingly, of the four animals examined after treatment with 0.52 MPa, two had no extravasations anywhere in the brain. In contrast, the brain subject to 5.0 MPa B-mode ultrasound had an average of 9.3 extravasations per slide (FIG. 19b where an example of severe red blood cell extravasation from the brain exposed to 5.0 MPa is shown). Since the B-mode was only applied to about ⅙ of the brain, this number under-represents the extravasation rate relative to the other two groups.

### Behavioral Assessments

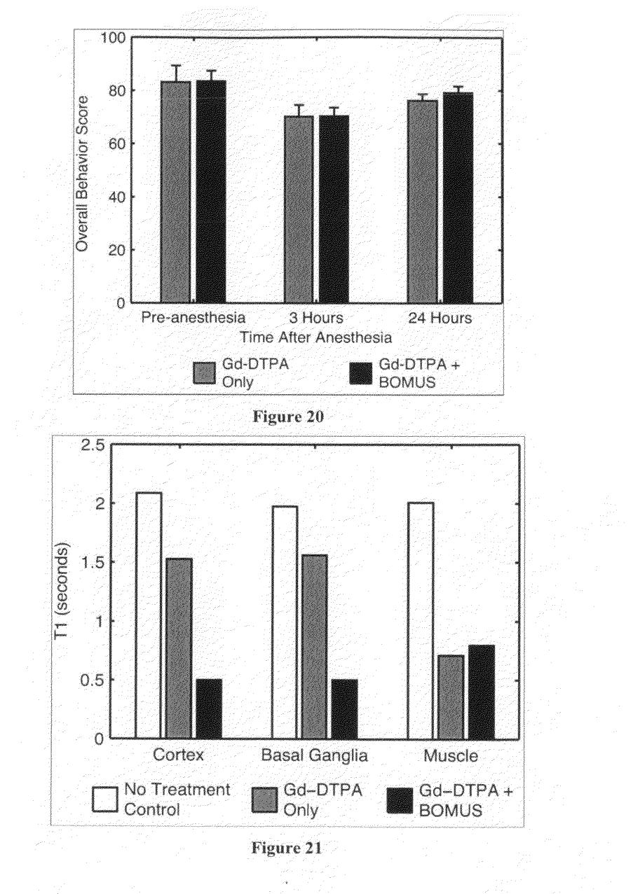

To determine if the BOMUS could potentially be used in longitudinal studies, behavioral assessments were performed on selected mice at three time points: prior to the experiment, 3 hours after the experiment (i.e., 3 hours after recovering from isoflurane anesthesia), and 24 hours after the experiment. Animals treated with BOMUS (0.36 MPa ultrasound pressure, 3.2 mmol/kg Gd-DTPA) were compared with control animals that were treated identically but did not receive ultrasound or microbubbles. The battery of 16 behavioral tests was performed and the scores summed to generate an overall behavior score (FIG. 20). FIG. 20 depicts results of behavioral testing before anesthesia and 3 and 24 hours after recovery from anesthesia. The average behavior (±SEM) score for control (n=3) and BOMUS (0.8 MPa) treated (n=8) animals is shown. Relative to the pre-anesthesia baseline, all animals show a decrease in behavior score 3 hours after anesthesia, but they largely recover by the next day. At each time point, no difference was seen between the two groups, indicating that BOMUS did not measurably affect animal behavior. For both groups, with respect to baseline, there was a decrease in the average behavior score 3 hours after anesthesia. This drop largely recovered (but not completely) by the 24-hour time point. However, at each of the three testing times, no difference was observed in the average behavior scores between the BOMUS-treated and control animals.

### T1 Estimation

To measure the change in relaxivity due to the Gd-DTPA, T1 was estimated in ROIs selected from the cortex, basal ganglia and muscle (FIG. 21). In the control animal receiving neither Gd-DTPA nor BOMUS, T1 values were long in the cortex (2.08 s), basal ganglia (1.97 s), and muscle (2.01 s). In the animal given only Gd-DTPA, the muscle T1 shortened dramatically (0.71 s); but T1 was only shortened modestly in the cortex (1.53 s) and basal ganglia (1.56 s) because the intact BBB excluded the Gd-DTPA. However, in the BOMUS-treated animal, Gd-DTPA not only shortened T1 in the muscle (0.80 s), but Gd-DTPA also crossed the BBB and dramatically shortened T1 in the cortex (0.50 s) and basal ganglia (0.50 s).

### High-Resolution MRI

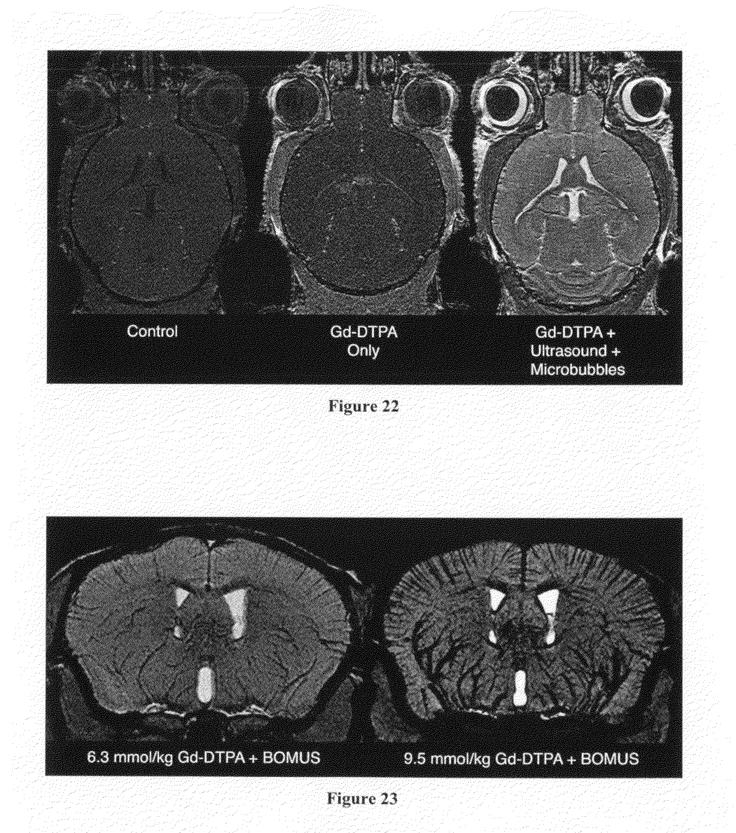

By taking advantage of the shortened T1 of the brain tissue, high-resolution (52×52×100 micrometers3) T1-weighted images were obtained (FIG. 22) from BOMUS-treated animals in only 51 minutes. For comparison, images of untreated, and Gd-DTPA-only mice were also acquired at the same resolution. (The control mouse receiving no contrast agent and the mouse receiving only Gd-DPTA have relatively low signal. The animal receiving Gd-DTPA along with BOMUS (microbubbles+ultrasound) shows an increase in SNR of 90% and 63% over the other two. (SNR measurements made in left anterior cortex.)) A fixed TR of 25 ms was used and the flip angle was adjusted for each scan to maximize the SNR in the brain. The images from the BOMUS-treated animals showed superior SNR and tissue contrast. For example, the layering in the hippocampus and cerebellum could not be distinguished in the control or Gd-DTPA-only mice, but this layering was clearly seen in the BOMUS-treated animals.

By increasing the dose of Gd-DTPA, it was also possible to obtain negative contrast vascular images (FIG. 23). FIG. 23 depicts minimum intensity projections of a 600-□m axial slab from SPGR images (high resolution protocol) from BOMUS-treated animals given high doses of Gd-DTPA. BOMUS allows the Gd-DTPA to enhance the parenchyma of the brain, but high concentration of Gd-DTPA in the blood stream causes susceptibility-induced loss of signal from the blood and perivascular tissue. This allows the delineation of cortical vessels (running perpendicular to the cortical surface). When the dose of Gd-DTPA is increased to 9.5 mmol/kg, this effect is exaggerated. These images were acquired using the high-resolution protocol in BOMUS-treated mice receiving either 6.3 or 9.5 mmol/kg Gd-DTPA. The background brain tissue is enhanced by the Gd-DTPA that has crossed the BBB. However, the large vascular content of Gd-DTPA causes susceptibility-related signal loss from the blood and perivascular tissue signal. This allows the delineation of both large and small vessels. For example, the relatively large branches of the middle cerebral arteries supplying the basal ganglia were clearly seen moving dorsally from the base of the brain. Many of these vessels are larger than 50 □m in diameter (28-30). However, in addition to these larger vessels, the cortical vessels that run perpendicular to the cortical surface can also be visualized. Previous work has indicated that these vessels are less than 50 micrometers in diameter (i.e., below the resolution of the image)(Ghaghada K B, Howles G P, Johnson G A, Mukundan S. High-resolution contrast enhanced magnetic resonance angiography of the mouse circle-of-willis. Proceedings of 16th Annual Meeting of ISMRM; 2008; Toronto; Howles G P, Ghaghada K B, Qi Y, Srinivasan Mukundan J, Johnson G A. High resolution magnetic resonance angiography in the mouse using a nanoparticle blood pool contrast agent. Magn Reson Med 2009; In Press; Dorr A, Sled J G, Kabani N. Three-dimensional cerebral vasculature of the CBA mouse brain: A magnetic resonance imaging and micro computed tomography study. Neuroimage 2007; 35(4):1409-1423). By taking advantage of the through-space susceptibility effect, these vessels can be detected even though they are smaller than the resolution of the image. While this susceptibility vascular imaging worked well with 6.3 mmol/kg Gd-DTPA, the effect was excessive when the dose was raised to 9.5 mmol/kg.

### Discussion

While there is great interest in studying the mammalian brain, such as the mouse brain, with MRI, long T1 and poor tissue contrast have been limiting. For ex vivo studies, staining the brain with contrast agents has enabled dramatic improvements in spatial resolution, tissue contrast, and scan time (Johnson G A, Ali-Sharief A, Badea A, Brandenburg J, Cofer G, Fubara B, Gewalt S, Hedlund L W, Upchurch L. High-throughput morphologic phenotyping of the mouse brain with magnetic resonance histology. Neuroimage 2007; 37(1):82-89). However, the BBB has interfered with the use contrast agents for in vivo studies. Here a method has been presented for contrast-enhanced imaging of the whole mouse brain using ultrasound to open the BBB. For researchers interested in contrast-enhanced brain imaging, the BOMUS technique has the following advantages over previous BBB disruption techniques: (a) fast and simple; (b) non-invasive and therefore suitable for in vivo and longitudinal studies; (c) global, opening both hemispheres.

The BOMUS technique presented here is fast and simple to perform. Animal preparation requires only a tail vein catheter and optionally a haircut, and the insonation takes only 3 minutes. While the precise calibration of the ultrasound pressure (described previously) did require a specialized hydrophone, the equipment required for BOMUS is all commercially available and requires limited expertise in ultrasound to assemble and use.

The BOMUS technique is non-invasive and reversible. In this study, mice were assessed not only for histological signs of damage, but also behavioral changes due to the procedure. In the data presented here (n=3), BOMUS with 0.36 MPa showed no red blood cell extravasations in the brain, and the mice recovered identically to those not receiving BOMUS. BOMUS and control animals showed no differences in behavior scores, but both groups showed slightly lower behavior scores 24 hours after anesthesia compared to baseline. This change may be due to residual anesthesia effects after 24 hours. Alternatively, this change in scores may be due to diurnal or environmental factors: the baseline test was performed during a more active time of day (early morning) and after exposure to a new environment (a new cage from the vivarium), while the 24-hour post-experiment test was performed during a less active time of day (afternoon) after the mice had acclimated to the cage.

While 0.36 MPa had no observed negative effects, 0.52 MPa BOMUS did cause a small number of extravasations in some of the animals. While the behavior of this group was not measured systematically, it was observed that after 0.52 MPa BOMUS, approximately 30% of the mice either died or failed to recover completely. Previous reports using focused ultrasound and microbubbles have regarded a few extravasations as an acceptable level of damage for a “non-invasive” technique (Hynynen K, McDannold N, Vykhodtseva N, Jolesz F A. Noninvasive MR imaging guided focal opening of the blood-brain barrier in rabbits. Radiology 2001; 220(3):640-646). While this may be true when BOMUS is applied to a very small region of the brain (2-3 mm), our observations indicate that when BOMUS is performed on the whole brain, acoustic pressures that are associated with occasional extravasations may affect the recovery of the animal. In light of this inconsistent recovery after 0.52 MPa, we conclude that an acoustic pressure that does not cause extravasation should be used in global BBB disruption.

In comparing our pressure measurements with those from previous reports using focused ultrasound, it should be noted that we report acoustic pressure that reaches the surface of the scalp at the center of the ultrasound beam. The beam profile data shown above demonstrate that the acoustic pressure towards the edge of the beam is only about 34% of the peak. Furthermore, acoustic attenuation through the mouse skull reduces the acoustic pressure reaching the brain by an estimated 25% (de-rating based on results presented by Choi et al. (Choi J J, Pernot M, Brown T R, Small S A, Konofagou E E. Spatio-temporal analysis of molecular delivery through the blood-brain barrier using focused ultrasound. Physics in Medicine and Biology 2007; 52(18):5509-5530)). This suggests consistent BBB disruption is obtained at peak-negative acoustic pressures ranging from 0.09 MPa to as little as 0.03 MPa. These pressures are much lower than the levels (typically 0.4 to 0.5 MPa) reported by others (McDannold N, Vykhodtseva N, Hynynen K. Use of ultrasound pulses combined with Definity for targeted blood-brain barrier disruption: A feasibility study. Ultrasound in Medicine and Biology 2007; 33(4):584-590). This reduced pressure threshold may be due to the higher dose of lipid microbubbles used in this work (approximately 1.2 ml/kg) compared to others using lipid microbubbles (10 microliters/kg). This explanation is supported by preliminary work in our lab and work by others (18), which suggest that large differences in levels of circulating microbubbles affect the acoustic pressure threshold for BBB disruption. While the dose we use is higher than the clinically recommended dose (10 microliters/kg), the data presented here did not reveal any negative effects at the higher dose.

We have demonstrated the utility of the BOMUS technique for “active staining” of the brain with Gd-DTPA in vivo. The reduction in T1 (from approximately 2000 ms to 500 ms) allowed high-resolution images (52×52×100 micrometers) to be obtained in only 51 minutes. The time-course data showed that this staining is stable for several hours, giving a long window for imaging, but washes out within a day. The staining provided excellent tissue contrast, which revealed features such as the layering of the hippocampus and cerebellum. Future work with other MRI contrast agents might reveal different patterns of tissue contrast.

In addition to administering anatomical contrast agents, the BOMUS technique has the potential to allow for the administration of functional and molecular contrast agents. Manganese has been used as a functional contrast agent that can distinguish neuronal activity. To administer manganese to the brain of rats, intracarotid mannitol infusions have been used to open the BBB, thus allowing functional imaging in a limited region of the rat brain. However, translating such a technique to mice has been challenging due to the technical difficulty and invasiveness of the mannitol procedure. The global BOMUS technique described here would not only enable such experiments in mice, but would also permit their use in high-throughput or longitudinal studies. Furthermore, the BOMUS technique would allow manganese to be administered to the whole brain, opening up new experimental possibilities (Howles-Banerji G P. Active staining for in vivo magnetic resonance microscopy of the mouse brain [dissertation]. Durham (NC): Duke University; 2009. 167 p).

Similarly, there is now an emergence of new molecular imaging agents for MRI and other modalities (Querol M, Bogdanov A. Amplification strategies in MR imaging: Activation and accumulation of sensing contrast agents (SCAs). J Magn Reson Imaging 2006; 24(5):971-982; Meade T J, Taylor A K, Bull S R. New magnetic resonance contrast agents as biochemical reporters. Curr Opin Neurobiol 2003; 13(5):597-602; Shapiro E M, Koretsky A P. Convertible manganese contrast for molecular and cellular MRI. Magnet Reson Med 2008; 60(2):265-269). Like existing contrast agents, nearly all of these new agents will be excluded by the BBB. BOMUS may enable the use of these new agents for studying mouse models of neurological disease. Recent work using focused ultrasound with microbubbles has demonstrated that both antibodies and molecular imaging agents may be administered using ultrasound-mediated BBB disruption (Raymond S B, Treat L H, Dewey J D, McDannold N J, Hynynen K, Bacskai B J. Ultrasound enhanced delivery of molecular imaging and therapeutic agents in Alzheimer's disease mouse models. PLoS ONE 2008; 3(5):e2175; Kinoshita M, McDannold N, Jolesz F A, Hynynen K. Targeted delivery of antibodies through the blood-brain barrier by MRI-guided focused ultrasound. Biochemical and Biophysical Research Communications 2006; 340(4):1085-1090).

### Conclusions

In this work, the blood-brain barrier was opened using unfocused ultrasound and microbubbles. This technique has several notable features: it (a) can be performed transcranially in mice; (b) takes only 3 minutes and uses only commercially available components; (c) opens the BBB throughout the brain; (d) causes no observed histological damage or changes in behavior; and (e) allows the BBB to be restored within 4 hours. Using this technique, Gd-DTPA was administered to the mouse brain parenchyma, thereby shortening T1 and enabling the acquisition of high-resolution (52×52×100 μm3) images in 51 minutes in vivo. By enabling the administration of imaging and therapeutic agents, this technique is a promising tool in the study mouse models of human neurological diseases.

### Example 2

### Blood-Brain Barrier (BBB) Disruption Using a Diagnostic Ultrasound Scanner

The objective of this example was to transcranially and nondestructively disrupt the BBB in the mouse using focused, diagnostic ultrasound and contrast agent, and to quantify that disruption using MRI and MR contrast agent. Each mouse was placed under isoflurane anesthesia and the hair on top of its skull was removed before treatment. A diagnostic ultrasound transducer was placed in a water bag coupled with gel to the mouse skull. Definity (US contrast) and Magnevist (MR contrast) were injected concurrent with the start of a custom ultrasound transmission sequence. The transducer was translated along the rostral-caudal axis to insonify three spatial locations (2 mm apart) along one half of the brain for each sequence. T1-weighted MR images were used to quantify the volume of tissue over which the BBB disruption allowed Magnevist to enter the brain, based upon increases in MR contrast-to-noise ratio (CNR) as compared to the noninsonified portions of the brain. Ultrasonic frequency, pressure, and pulse duration, as well as Definity concentration and injection time were varied. Preliminary results suggest a threshold for BBB opening with increased pressure and pulse duration (consistent with literature performed at lower frequencies). A range of typical diagnostic frequencies (e.g. 5-8 MHz) generated BBB disruption. Comparable BBB opening was noted with varied delays between Definity injection and insonification (0-2 min) nor Definity concentrations (400-2400 μL/kg). Standard B-mode imaging (MI=1.5, duty cycle=0.4%) was associated with blood cell extravasation as determined by histological evaluation; however, minimal damage was noted after the low-pressure, custom sequences (MI≦0.65). This study has shown the ability of a diagnostic ultrasound system, in conjunction with Definity, to open the blood brain barrier transcranially in a mouse model for molecules approximately 1 kDa in size. Opening was achieved at higher frequencies than previously reported and was localized under ultrasound image guidance. A typical, ultrasound imaging mode (PW Doppler) with specific settings (transmit frequency=5.7 MHz, gate size=15 mm, pulse repetition frequency=100 Hz, system power=15%) successfully opened the BBB, which facilitates implementation on any commercial, scanner. Localized opening of the blood-brain barrier may have potential clinical utility for the delivery of diagnostic or therapeutic agents to the brain.

### Methods

Animal Setup: Thirty-six C57BL/6J mice (20-27 g) were used in this study. Each mouse was anesthetized with isoflurane and the scalp depilated. An IV tail catheter for perflutren lipid microspheres (Definity®, Bristol-Myers Squibb Medical Imaging, N. Billerica, Mass., USA) injection and an IP catheter for gadopentetate dimeglumine (Magnevist R, Bayer Schering Pharma, Berlin, Germany) injection were put in place. A thin plastic bag containing a 17 mm water path was coupled to the scalp with ultrasound gel. A hemicylindrical plastic shell was placed over the thorax of the mouse to prevent the weight of the water from adversely affecting breathing. A Visualsonics stereotaxic positioning system (Vevo Integrated Rail System, Toronto, Canada) was used to center the B-mode image in the transverse plane through the eyes.

### Ultrasound Application

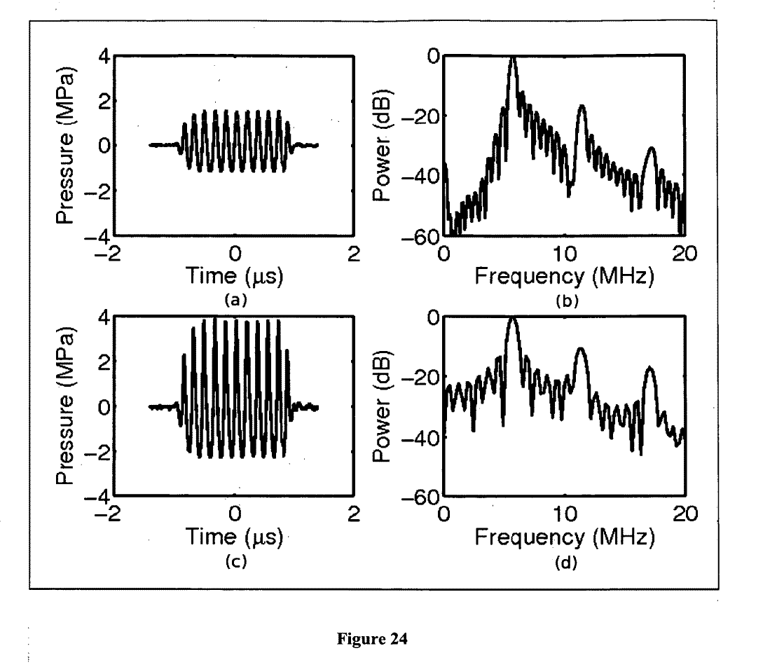

A Siemens Sonoline™ Antares diagnostic scanner and VF10-5 transducer (Siemens Medical Solutions USA, Inc., Issaquah, W A) were used to insonify the mouse brain approximately 3 mm deep to the dorsal surface of the skull using a transducer focal depth of 2 cm (for both electronic focusing in azimuth and the lens focus in elevation; a water path was used as a standoff to this depth). All acoustic pressure measurements were made with a Sonora SN S4-251 hydrophone with a 0.4-mm spot size membrane (Sonora Medical Systems, Inc., Longmont, Colo.) and are reported in water (no derating). A baseline sequence with 20 ms, 2.72±0.03 MPa (peak-to-peak) pulses repeated at 10 Hz for 30 seconds was implemented based on (Choi et al, 2007). FIG. 24 shows typical pulses used for this study. Specifically, FIG. 24 shows example waveforms (a,c) and power spectra (b,d) of pulses with peak-to-peak pressures of 2.72 MPa (a,b) and 6.16 MPa (c,d). At these pressures, the waveforms demonstrate some nonlinearity. The corresponding MI (P−0.3/√f) are 0.33 and 0.65, respectively. Modulation of the ultrasonic sequence as well as the dosage and timing of the Definity injection was performed. Ultrasonic parameters were investigated by varying pulse durations between 0.35 μs and 20 ms, peak-to-peak pressures between 1.05±0.06 and 6.16±0.02 MPa, and frequencies between 5 and 8 MHz. Definity doses between 10 and 60 μL (400-2400 μL/kg) and Definity injection times were also briefly examined, ranging from start of insonification to 2 minutes prior to insonification. Table 1 summarizes the exposure parameters investigated in this study along with the number of insonifications evaluated for each set of parameters. Each location was insonified for 30 seconds with a PRF of 10 Hz and an unapodized, F/1.5 configuration except the PW Doppler sequence (*) which used an 100 Hz PRF and an apodized, F/4 configuration. Sequences above the double line are presented in the plots herein, while those below serve as discussion points.

| | Definity | Delay after | | | Pulse |
|---|---|---|---|---|---|
| Number of | Dosage | Definity | Frequency | Pressure | Duration |
| Sonications | (μL) | injection (s) | (MHz) | (MPa) | (ms) |
| 4 | 30 | 0 | 5.7 | 1.05 | 20 |
| 4 | 30 | 0 | 5.7 | 6.16 | 20 |
| 4 | 30 | 0 | 5.0 | 2.27 | 20 |
| 4 | 30 | 0 | 5.7 | 2.72 | 20 |
| 4 | 30 | 0 | 6.7 | 3.84 | 20 |
| 4 | 30 | 0 | 8.0 | 5.20 | 20 |
| 4 | 30 | 0 | 5.7 | 2.72 | 2.0e−3 |
| 4 | 30 | 0 | 5.7 | 2.72 | 7.0e−2 |
| 2 | 10 | 0 | 5.7 | 6.16 | 20 |
| 2 | 60 | 0 | 5.7 | 6.16 | 20 |
| 2 | 30 | 7 | 5.7 | 6.16 | 20 |
| 2 | 30 | 60 | 5.7 | 6.16 | 20 |
| 1 | 30 | 120 | 5.7 | 6.16 | 20 |
| 2 | 30 | 0 | 6.7 | 2.75 | 20 |
| 2 | 30 | 0 | 8.0 | 2.26 | 20 |
| 1 | 30 | 0 | 5.7 | 1.60 | 20 |
| 1 | 30 | 0 | 5.7 | 3.80 | 20 |
| 1 | 30 | 0 | 5.7 | 2.72 | 3.5e−4 |
| *5 | 30 | 0 | 5.7 | 2.72 | 7.0e−3 |

### BBB Opening Procedure

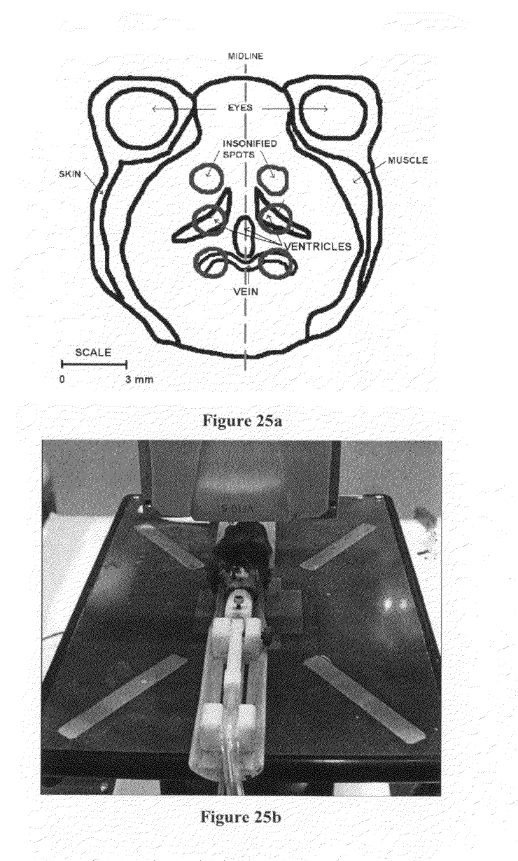

For opening the BBB, two different ultrasound sequences (selected from those shown in Table 1) were tested on each animal—one on each side of the brain—to reduce the number of animals sacrificed for these experiments. For each sequence, three different locations were serially insonified 2 mm apart in the rostral-caudal direction (see FIG. 25 which shows (a) Anatomical sketch of a coronal slice of the brain with the insonification spots. Only the two most rostral spot positions were analyzed in the MR images. (b) Setup and transducer orientation relative to the mouse. Note: The water bag is not shown in FIG. 25). Using B-mode, ultrasound image guidance and the stereotaxic positioning system, the transducer was moved to the first location: 3 mm posterior to the eyes and 1.5 mm to the left of the midline as shown in FIG. 25. At the onset of a 30 second ultrasound sequence, Magnevist (6.3 mmol/kg IP) and Definity (30 μL IV) were injected. (We have found that this dose of Magnevist produces a consistent level of enhancement in mice.) After the 30-second sequence was finished, the transducer was then translated such that two more focal spots were insonified 2 and 4 mm posterior to the first spot at 1 and 2 minutes after the Definity injection (only one injection per side), respectively. Prior to administering the second sequence (right side of brain), an IV saline flush was given, and the Definity was allowed to clear over 5 minutes. The half-life for Definity in blood is reported to be only a 1.3 minutes (Unger et al, 2004; Def, 2004), which is consistent with qualitative observations in this work. The same procedure was then repeated with a different sequence 1.5 mm to the right of the midline but without reinjection of Magnevist, which clears slowly with a half-life of 1.6 hours (Mag, 2008).

Because Magnevist is normally excluded by the BBB, our assay for BBB disruption was to monitor the signal enhancement in MR images. After insonification of all six locations, the animal was placed in a quadrature, 300.5 MHz birdcage coil (M2M Imaging, Cleveland, Ohio, USA) tunable for mice (20-30 grams) and imaged in a 7T MRI system interfaced to a GE EXCITE console. A 3D spoiled gradient recalled echo (SPGR) sequence was used to acquire T1-weighted images approximately 30 minutes after insonification of the first spot. Because Magnevist is normally excluded by the BBB, regions of brain enhancement in the T1-weighted images were interpreted as regions of BBB disruption.

### Image Analysis

Image registration between the ultrasound and MR images was performed by aligning a control point defined at the top of the skull directly above the center of the BBB opening from left to right in the MR images and along the beam index used for BBB opening in the ultrasound image. The hyper-intense structures in the ultrasound image, corresponding to bones in this study, were then overlaid onto the corresponding MR images to evaluate the effectiveness of the ultrasonic guidance.

The degree of opening was evaluated by semi-automatic segmentation of the volumes of enhanced brain tissue in the MR images. By inverting the gray-scale values in the MR image, applying a 3-D watershed algorithm (The MathWorks, Inc., Natick, Mass.), and ignoring any voxels originally below the background level, the contrast-enhanced volumes associated with BBB opening were segmented. The full-width half-maximum (FWHM) contours in each slice of a volume were then used to calculate the mean gray-level as well as the dimensions and total volume for each opened region, or spot, in the brain. If the contralateral region of the brain to the region of interest was not insonified, the unopened BBB level was calculated as the mean gray-level in the opposite hemisphere; otherwise, the mean level in an unopened region of equivalent size and shape a few millimeters lateral and caudal to the opened region was used. The contrast-to-noise ratio (CNR) was calculated as the difference in mean gray-levels of the FWHM-defined volumes for the opened and unopened BBB regions divided by the standard deviation in an empty region of the MR image (no tissue present); therefore, a higher CNR is indicative of more BBB opening (or Magnevist in the brain tissue) and a CNR of 0 indicates no discernible opening.

### Histology

The brains of nine mice were processed for histology. Eight of these mice were insonified with the most aggressive sequence intentionally used for BBB opening (5.71 MHz, 20-ms pulses, 10 Hz PRF, 6.17 MPaₚₚ, 30 μL Definity) in at least one location. Insonification with less aggressive BBB sequences was also performed in these brains, as described previously. In the ninth brain, the effect of a commercial, B-mode sequence on the brain was evaluated. The tissue of these mice was fixed using transcardiac formalin (10%) perfusion. Coronal sections of the excised brains were taken at 0.5 mm intervals (at least 17 slices per animal) and stained with hematoxylin-eosin. These sections were examined for evidence of red blood cell extravasation into the brain parenchyma, which has been reported to be the first sign of tissue damage (Burkitt et al, 1996; Hynynen et al, 2005).

### Results

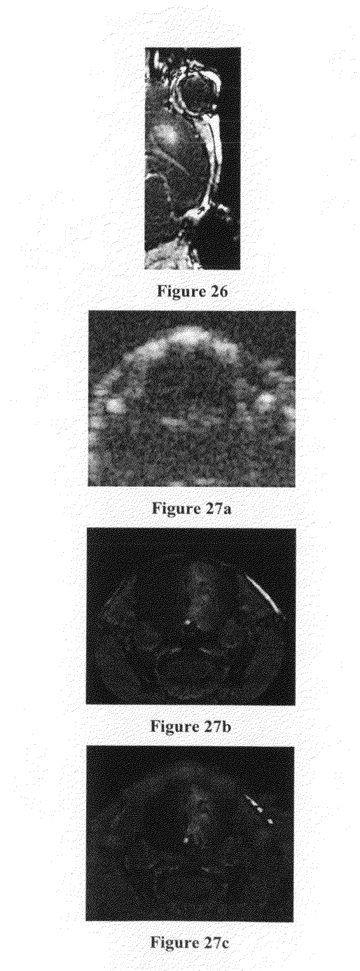

The blood-choroid barrier of the ventricles opened more readily than the blood-brain barrier. As shown in FIG. 26, only one of the three insonification spots is visible in brain tissue, but the ventricles are clearly visible. FIG. 26 depicts BBB opening with PW Doppler. 5.7-MHz, 7-μs ultrasound pulses repeated at 100 Hz with an apodized F/4 configuration yielding 2.72 MPaₚₚ were transmitted for 30 seconds immediately after a 30-4, Definity injection. Furthermore, because the ventricles are interconnected, an opening of the blood-choroid barrier in one part of the brain caused enhancement throughout the ventricular network. As a result, quantitative measurements are only reported for the most rostral spot (spot 1) for each 3 spot parameter set, because the ventricles were not present within this region (FIG. 25).

The stereotaxic stage in conjunction with ultrasound image guidance prior to injection of Definity made repeatable localization within the brain efficient. The midline between the eyes was easily visible in B-mode images and the stereotaxic positioning system could be moved such that the BBB opening insonification accurately occurred 3 mm caudal and 1.5 mm lateral to this point. As demonstrated in FIG. 27, the maximum contrast in the BBB opening was accurate in the medial-lateral and ventral-dorsal axes. FIG. 27 depicts images showing a) B-mode ultrasound only (5.7 MHz), b) MR only, and c) structures seen in ultrasound (found by thresholding) overlaid in red on the MR image. The yellow + shows the intended center of the ultrasound focus based on the B-mode image. The white region surrounding the + on the right side of the MR image is indicative of T1 enhancement from Magnevist crossing the BBB. BBB opening was achieved using 5.7-MHz, 20-ms ultrasound pulses repeated at 10 Hz with an F/1.5 configuration, yielding pressures of 6.16 MPaₚₚ, in a 30-second insonification immediately after a 30-μL Definity injection.

The general impact of varying the amount of Definity present during insonification was considered in two ways: (1) increasing the dose and (2) changing the time in circulation before insonification. BBB opening with similar CNR was seen with an increasing dose of Definity from 10 to 60 pt. The impact of varying delays between Definity injection and ultrasound initiation were observed over a range of times. For some experimental configurations, it may be hard to have concurrent injection and insonification initiation; therefore, a fast, but reasonable, range of delays between 0 and 2 minutes were considered. Opening occurred in all cases, with slightly higher CNRs observed with no delay.

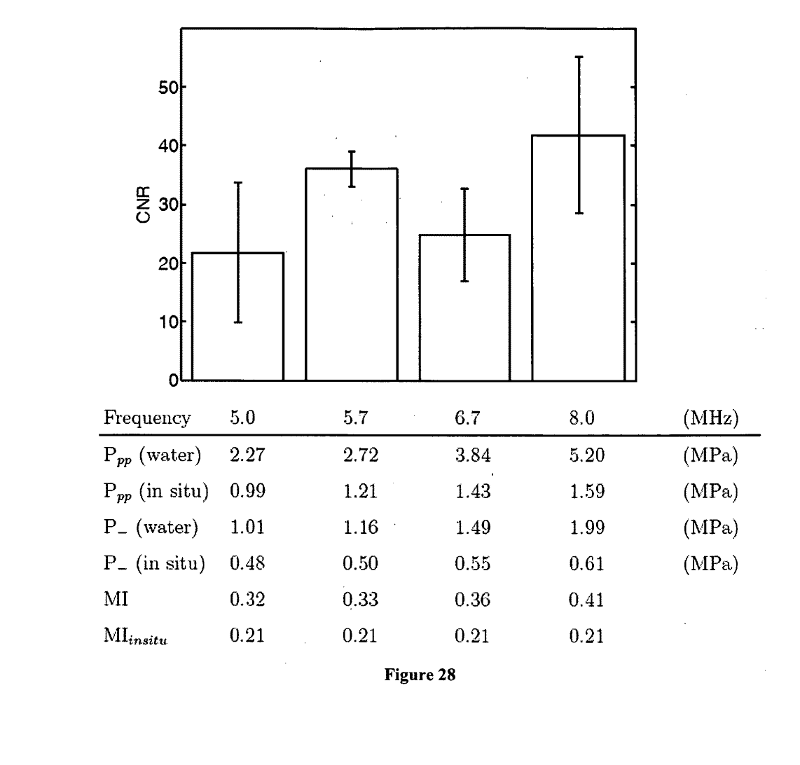

Previous BBB opening studies have looked at frequencies below 2.04 MHz, but the bandwidths of diagnostic transducers are usually centered around higher frequencies. Therefore, frequencies of 5, 5.7, 6.7 and 8 MHz were tested with equal M_{in situ} (0.21, peak negative in situ pressure over the square root of frequency (McDannold et al, 2008a)). BBB disruption was generated at each of these frequencies with insignificant differences in CNR (p>0.05), as shown in FIG. 28. FIG. 28 depicts BBB opening for ultrasonic transmission frequencies from 5 to 8 MHz for the same system input voltage. 20-ms ultrasound pulses repeated at 10 Hz with an F/1.5 focal configuration were transmitted for 30 seconds immediately after a 30-μL Definity injection. The mean and standard deviation for four animals are indicated at each frequency. Non-derated and derated pressures as well as MI (P−0.3/√f) and M_{in situ} (P−in situ/√f) for each frequency are listed. The standard deviation of these pressure measurements are ≦1%. The acoustic outputs for the frequencies tested are also shown. The values measured in water and derated by the attenuation of the skull and intervening brain tissue (attenuation values reported in (Choi et al, 2007; Duck, 1990)), as well the MI (peak negative pressure derated by 0.3 dB/cm/MHz over the square root of frequency) (NCR, 2002) and estimated M_{in situ} values are reported. It was noted in preliminary studies that when the MI_{in situ} at 6.67 MHz was lowered to 0.17, BBB disruption was still easily seen; however, when the W_{in situ} at 8.0 MHz was lowered to 0.10, no BBB disruption was seen.

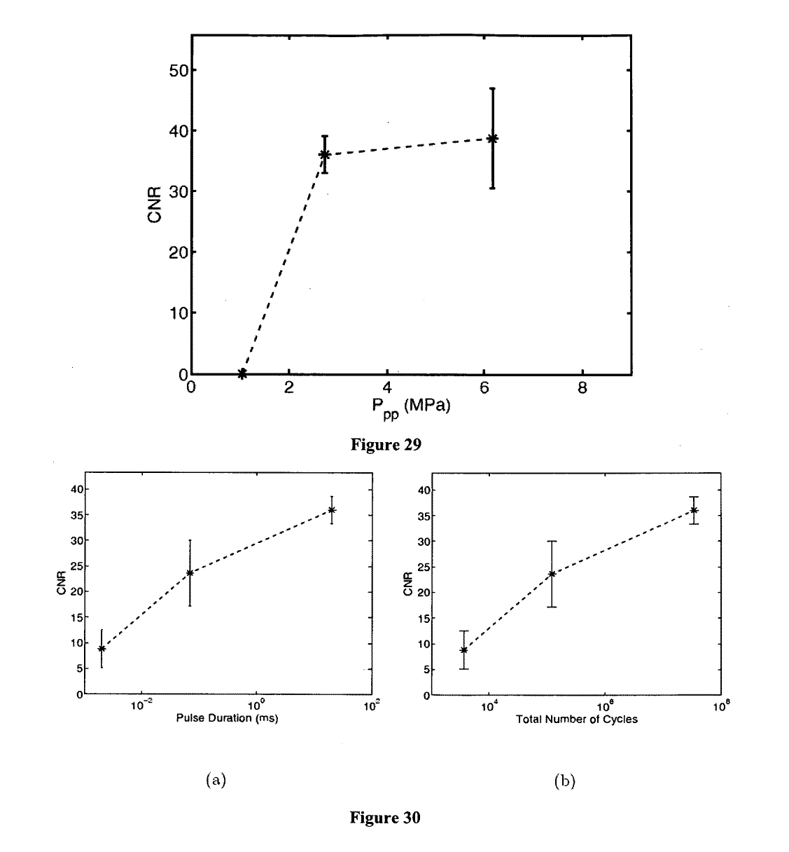

Regardless of the mechanism, most acoustic bioeffects are related to the energy delivered to the region of interest and duration of insonification. Therefore, we evaluated the effects of changing pressure and pulse duration on the degree of BBB opening. While maintaining a constant frequency (5.7 MHz) and changing the pressure, visible opening was shown to require a peak-to-peak pressure exceeding a threshold between 1.05±0.01 MPa and 2.72±0.01 MPa, as shown in FIG. 29. FIG. 29 depicts the effect of ultrasonic pressures from 1.05 to 6.16 MPaₚₚ (non-derated) on BBB opening. 5.7-MHz, 20-ms ultrasound pulses repeated at 10 Hz with an F/1.5 configuration were transmitted for 30 seconds immediately after a 30-μL Definity injection. The mean and standard deviation for four animals is given at each pressure. Above 2.72±0.01 MPa, the increase in contrast was insignificant (p>0.05). A single case from each of two intermediate pressure values (1.60 and 3.80 MPaₚₚ) resulted in CNR values (24 and 39) within the appropriate ranges, as determined by FIG. 29.

In order to show the feasibility of BBB opening with a clinical scanner, a range of pulse durations corresponding to Doppler and acoustic radiation force impulse (ARFI) imaging were evaluated (see Table 1) and compared with a 20-ms pulse previously shown to open the BBB (Choi et al, 2007), all at a pulse repetition frequency (PRF) of 10 Hz and a total insonification time of 30 seconds. The opening for 2-μs pulses was not always clear without a priori knowledge of the expected location of BBB disruption. However, pulse durations of 70 μs and 20 ms were clearly visible, as evidenced by the CNR values in FIG. 30 (semi-log plots in x). FIG. 30 depicts a) Effect of pulse durations of 0.35 μs (B-mode), 2 μs (Color Doppler), 70 μs (Acoustic Radiation Force Impulse Imaging), and 20 ms on BBB opening. 5.7-MHz ultrasound pulses repeated at 10 Hz with an F/1.5 configuration yielding 2.72 MPaₚₚ were transmitted for 30 seconds immediately after a 30-μL Definity injection. The mean and standard deviation for four animals is given at each pulse duration. b) Same data presented as a function of the total number of cycles in the insonification sequence. Note these are semi-log plots in x. For this configuration, the threshold for uniform, well visible (CNR>10) opening is a pulse duration near 2 μs, repeated such that the total number of cycles exceeds 10⁵. It should also be noted that in a single case where a 2-cycle B-mode pulse (0.35 μs) was used with all other parameters the same (e.g., 2.72 MPaₚₚ), no opening was seen.

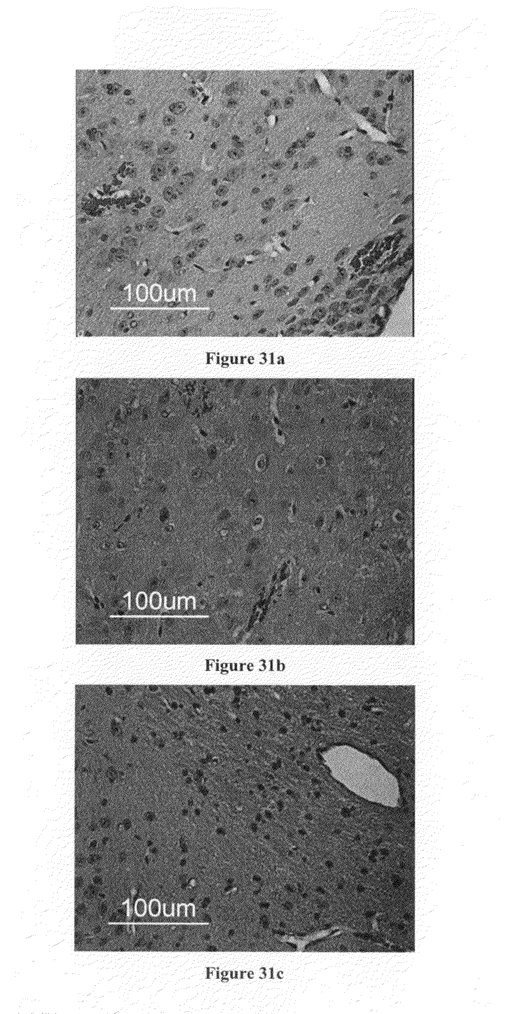

In the preliminary work for this study, standard B-mode insonification (MI=1.5, as defined by (AIU, 1992)) with Definity present was found to open wide planes of BBB and to result in significant blood cell extravasation (139 sites in one brain), as evidenced in FIG. 31. FIG. 31 depicts H & E stained histology of a) blood cell extravasation caused by standard B-mode (MI=1.5, 0.35 μs, 5.7 MHz, 34.60 MPaₚₚ insonifying for five 30 second periods at a 36 Hz frame rate with 30-μL Definity) as well as b) extravasated (top) and vessel enclosed (bottom) blood cells and c) no damage with the most aggressive experimental ultrasound exposure used for this study (MI=0.65, 5.7-MHz transmit frequency, 6.17-MPaₚₚ pressure (in water), F/1.5, and 20-ms pulse duration with 30-μL Definity.). Therefore, for all other data, B-mode images were only acquired prior to Definity injection. The histologic data from mice insonified with most aggressive, experimental ultrasound regime (MI=0.65, 5.7 MHz transmit frequency, 6.17-MPa peak-to-peak pressure (in water), F/1.5, 20 ms pulse duration, 3.42e7 total cycles, and a 10-Hz PRF) resulted in an average of 2.6±2.9 extravasated sites per brain (over 8 entire brains evaluated). Because not all of the extravasations seen were near an intended sonication location, it is not clear whether the small amount of blood cell extravasation was a function of the insonification or the perfusion, fixation, and sectioning methods.

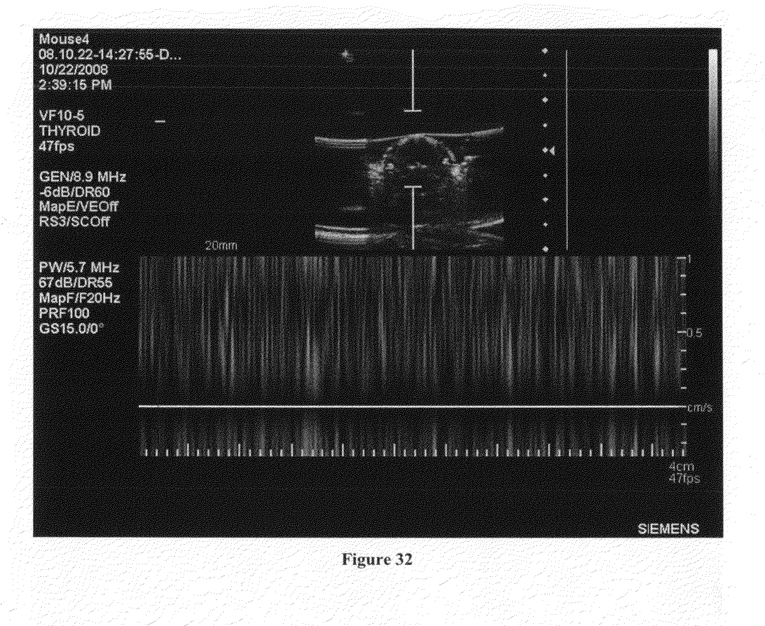

Based on the range of pulse durations and pressures that resulted in obvious opening (CNR>10) presented here, it became evident that a pulsed Doppler sequence could be utilized for BBB opening in the mouse. As a proof of concept, the VF10-5 transducer was placed in the standard, clinical, pulsed wave (PW) Doppler mode (B-mode imaging frozen) on the Antares system with a frequency of 5.7 MHz, gate size of 15 mm, PRF of 100 Hz, and a system power of 15%, as indicated on the scanner monitor, for 30 seconds (see FIG. 32). FIG. 32 depicts example of image guidance and system settings for PW Doppler mode BBB opening. These settings resulted in a pulse duration of 7 μs and 1.2×10⁵ total cycles with an apodized F/4 focal configuration. The MI and peak-to-peak pressure of this configuration were equal to one of the standard configurations tested in this study (MI=0.33, 2.72±0.01 MPaₚₚ, CNR=24±7). As shown in FIG. 26, this sequence easily opened the BBB (CNR=21±9).

### Discussion

Visualization of the skull, zygomatic arches, and eyes in B-mode images made 3-D localization with the stereotaxic positioning system simple, fast, and repeatable. As evidenced by the registration of B-mode to MR images (FIG. 27), the location of peak opening was close to the focal point shown on B-mode. The center of the visible opening was not centered around this focus in the ventral-dorsal direction because the focus of the ultrasound beam was closer to the top of the skull. Furthermore, our studies indicated a change in the depth of the opening (center and dorsal-ventral extent) with anatomical position in the brain (rostral-caudal and left-right). Variable thickness in the skull and confounding effects from the ventricles (where the blood-choroid barrier is easier to open) may explain the variations with position. With a higher attenuation and speed of sound than tissue, variable thicknesses in the skull lead to changes in the pressure delivered in vivo due to increased attenuation and phase aberration (Tanter et al, 1998).

This study suggests that doses of Definity exceeding the manufacturer's clinical recommendations (10 μL/kg (Def, 2004)) can be given with only minimal histologic signs of damage to the mouse brain. Because it is difficult to administer the clinical doses for the small body weight of a mouse, the doses used in this study were in the range of 400 to 2400 μL/kg (bolus injection). BBB opening was achieved at all studied doses with similar CNRs. These results are consistent with those of another group using focused, ultrasound (0.69 MHz in rabbits) with Optison at lower doses (50-250 μL/kg) (McDannold et al, 2008b).

Previous work in mice demonstrated the need for increased pressure (near 3-fold) to observe BBB opening when there was a 15-minute versus a 1-minute delay between contrast agent (Optison) injection and insonification (intact skull, 1.5 MHz, 20-ms pulses at 10 Hz for 30 seconds, 400 μL/kg of Optison) (Choi et al, 2007). Minimal variation in opening for up to a 2 minute delay between injection and the start of insonification was observed in our studies. However, these data do suggest (without statistical significance) that starting the ultrasound insonification at exactly the same time as Definity injection may be optimal. To ensure that the most Definity possible is insonified before it is cleared or degraded by the system, it may be optimal to initiate insonification prior to injection.

A midrange subset of typical diagnostic frequencies was evaluated in this study to show the potential for using diagnostic scanners for BBB opening. A couple of factors, the in situ pressure and the resonance frequency of Definity, could influence the BBB opening observed at a given frequency for a constant pulse duration and insonification time. Of these two factors, the in situ pressure was directly evaluated and had an interesting impact on the BBB opening observed. At 5.7 MHz, there was a significant (p<0.05) change in CNR between 1.05 and 2.72 MPaₚₚ and an insignificant change between 2.72 and 6.16 MPaₚₚ. By assuming linear attenuation and accounting for acoustic loss through the skull (as reported by (Choi et al, 2007) at 1.5 MHz) and brain (Duck, 1990), the in situ pressures shown in FIG. 28 result. These pressures are indicative of the estimated increase in attenuation with frequency. Distortions of the beam due to phase aberration effects have also been shown to increase with frequency (Nock et al, 1989) and, therefore, may have further reduced the actual in situ pressure due to defocusing of the beam.

The second factor to consider is the resonance frequency of the Definity microbubbles. The mean bubble diameter of Definity, as described by the manufacturer, is between 1.1 and 3.3 (Def, 2004). According to Goertz et al., a lipid encapsulated bubble of those dimensions will have resonance frequencies ranging from about 13 to 3 MHz, respectively. A 2.2 μm diameter bubble (median of Definity diameters) with minimal damping should resonate around 4 to 5 MHz according to simulations (Goertz et al, 2003); however, as bubbles travel through the vasculature, this frequency decreases in vessels of smaller radii (e.g. capillaries) and further decreases near the center (lengthwise) of these smaller vessels (Sassaroli and Hynynen, 2004, 2005). Therefore, the bubbles themselves may bias the degree of BBB opening toward lower frequencies.

The combined impact of pressure and frequency on bubble dynamics is included in the mechanical index, which indicates lower frequency insonifications result in an increased likelihood for cavitation (Apfel and Holland, 1991). McDannold et al (2008a) reported recently that the threshold for BBB disruption is constant with a variant of mechanical index, MI_{in situ}. In our data, an MI_{in situ} of 0.10 at 8 MHz did not open the BBB, while an MI_{in situ} of 0.17 at 6.7 MHz and 0.21 at 8.0 MHz did. This is lower than McDannold's threshold of 0.46 in rabbits with 504/kg of Optison injected 10 seconds prior to insonification, though those experiments were performed post-craniotomy to eliminate skull aberrations (McDannold et al, 2008a). The order-of-magnitude higher concentration and/or type of ultrasonic contrast agent (Definity instead of Optison) may explain the lower MI_{in situ} threshold required for BBB disruption reported here. Other possible reasons might include different sonication conditions and animal models from McDannold et al (2008a).

Other possible mechanisms for BBB opening can be hypothesized based on the pulse duration studies. As with ultrasonic pressure, there appears to be a threshold for pulse duration (at a given pulse repetition frequency (PRF) of 10 Hz and total insonification time of 30 sec) that must be exceeded in order to observe appreciable BBB opening (CNR>10, FIG. 30) for the low pressures used in the majority of this study (≦6.16 MPaₚₚ in water, MI<0.65) which do not lead to significant tissue damage. At these low pressures, a pulse length of 2 μs, which is typical for diagnostic Color Doppler pulses, resulted in some opening with a low CNR (9±4). However, when B-mode (0.35-μs) pulses with a high MI (1.5) were used, easily visible opening was seen but it was associated with blood cell extravasation. Therefore, longer pulses with lower pressures were found to be preferred for BBB opening without blood cell extravasation. The fact that low pressures are effective is consistent with the hypothesis that inertial cavitation is not necessary for BBB opening (Fowlkes et al, 2008; McDannold et al, 2006).

These pulse duration studies also suggest that acoustic radiation force may be involved in the mechanism for BBB opening. Primary acoustic radiation force is proportional to acoustic temporal-average intensity (Dayton et al, 1997). In this study, a significant (p<0.05) increase in visible opening was observed between 2-μs (Ispta=1.1 mW/cm2), 70-μs (Ispta=39.5 mW/cm2), and 20-ms (Ispta=11.3 W/cm2) low pressure (2.72 MPaₚₚ) pulses at the same PRF and total insonification time, supporting the hypothesis that increased primary radiation force results in more BBB opening (Raymond et al, 2007). Although not monitored herein, these increased pulse durations would also be providing a longer time period for driving stable cavitation (i.e. bubble resonance without violent rupture) to open the BBB, as described in the literature for thrombolysis (Datta et al, 2008). Given the data presented here, the total number of cycles deposited at the focus for a given frequency may be a good indicator of the degree of BBB opening (CNR) observed. Two sequences with different pulse lengths and repetition frequencies, but the same total number of cycles, had similar CNRs, whereas the same pulse length but fewer cycles did not. Specifically, the PW Doppler sequence with 7-μs pulses at a 100 Hz PRF and a total number of cycles of 1.2e5 (Ispta=39.5 mW/cm2) had a CNR of 21±9. Based upon the Color Doppler (2-μs pulses, 3.4e3 total cycles, Ispta=1.1 mW/cm2, CNR=9±4) and ARFI pulse length's (70-μs pulses, 1.2e5 total cycles, Ispta=39.5 mW/cm2, CNR=24±8) investigated using a PRF of 10 Hz (FIG. 32), the PW Doppler sequence was expected to have a CNR of approximately 10. However, the difference in CNR between two sequences with the same total number of cycles and pressure at the focus, the PW Doppler sequence and acoustic radiation force pulses at 10 Hz, was insignificant (p>0.05). Conversely, previous studies by McDannold et al. showed no significant change in MR signal intensity while increasing the PRF from 0.5 to 5 Hz which resulted in an increase in the total number of cycles between 6.9e4 and 6.9e5 (McDannold et al, 2008b). These comparisons warrant further investigation into the relationship between the total number of cycles, pulse duration, and PRF in order to fully understand their impact on the degree of BBB opening. Furthermore, the vasoconstriction observed by Raymond et al (2007) may reduce the added effectiveness of BBB opening for long insonification times as decreased perfusion reduces the transport of microbubbles through the acoustic field.

Transcranial opening of the BBB with a diagnostic system was proven feasible in mice; however, significant barriers exist for extending this to humans. The mouse skull is very thin resulting in minimal acoustic loss due to attenuation (18% of the pressure amplitude at 1.525 MHz (Choi et al, 2007)) and phase aberration. Increased attenuation and defocusing due to thicker skulls in larger animals would require more acoustic output from the transducer to achieve the necessary in situ pressures. Although diagnostic systems may be capable of the necessary output, it may lead to excessive skull heating unless aberration correction or other techniques are employed (Clement et al, 2005; Aubry et al, 2003). However, there could be intra-operative situations in which a whole or partial craniotomy has already been performed and similar methods to those presented herein could be applied directly to the brain, but with the use of a more clinically relevant Definity dosage. A post-operative situation in which an acoustically transparent window has been implanted might also be feasible.

### Conclusion

The results of this study demonstrate the feasibility of BBB opening in mice with a commercial, diagnostic system and ultrasound contrast agent. Ultrasound at a frequency capable of imaging relevant anatomical landmarks in the mouse skull was successfully utilized both to image the mouse brain and to open the BBB in the presence of ultrasound contrast agent. Longer duration pulses (greater than or equal to 2 μs over a 30-second insonification time, at PRFs of 10-100 Hz, for a total number of cycles from ˜10⁵ to 10⁸) with low pressure amplitudes (1.6 to 6.2 MPaₚₚ, MI≦0.65) were found to allow MR contrast agent to enter the brain with minimal blood cell extravasation. B-mode also opened the BBB but resulted in significant blood cell extravasation. However, by using standard, system settings with a low MI (e.g., PW Doppler, 15% power, maximum gate size), the BBB was successfully opened without damage. The results of this study can be used to gauge the potential of other custom sequences or existing diagnostic regimes for studies to locally deliver drugs or other therapeutic agents through the BBB.

### REFERENCES

Acoustic Output Measurement and Labeling Standard for Diagnostic Ultrasound Equipment. American Institute of Ultrasound in Medicine, Rockville, Md. 1992. Exposure Criteria for Medical Diagnostic Ultrasound: II. Criteria Based on All Known Mechanisms. National Council on Radiation Protection and Measurements, Bethesda, Md. 2002. Definity R Vial for (Perflutren Lipid Microsphere) Injectable Suspension. Package insert. Bristol-Myers Squibb Medical Imaging, North Billerica, Mass. September 2004. Magnevist R (brand of gadopentetate dimeglumine) Injection. Package insert. Berlex Imaging, Montville, N.J. 2008. Apfel R, Holland C. Gauging the likelihood of cavitation from short-pulse, low-duty cycle diagnostic ultrasound. 1991; 17:179-185. Aubry J, Tanter M, Pernot M, Thomas J, Fink M. Experimental demonstration of noninvasive transskull adaptive focusing based on prior computed tomography scans. 2003; 113(1):84-93. Ballabh P, Braun A, Nedergaard M. The blood-brain barrier: an overview; structure, regulation, and clinical implications. Neurobiology of Disease. 2004; 16:1-13. Burkitt H, Stevens A, Lowe J, Young B, Wheater's Basic Histopathology: A Colour Atlas and Text, 3rd Edition. Churchill Livingstone, New York. 1996. Cho C W, Liu Y, Cobb W, Henthorn T, Lillehei K, Christians U, Ng K Y. Ultrasoundinduced mild hyperthermia as a novel approach to increase drug uptake in brain microvessel endothelial cells. Pharmaceutical Research. 2002; 19(8):1123-1129. Choi J, Pernot M, Small S, Konofagou E. Noninvasive, transcranial and localized opening of the blood-brain barrier using focused ultrasound in mice. 2007; 33(1):95-104. Clement G, White P, King R, McDannold N, Hynynen K. A magnetic resonance imagingcompatible, large-scale array for trans-skull ultrasound surgery and therapy. J Ultra-23 sound Med. 2005; 24:1117-1125. Datta S, Coussios C C, Ammi A, Mast D, De Courten-Myers G, Holland C. Ultrasound-enhanced thrombolysis using Definity R as a cavitation nucleation agent. 2008; 34(9):1421-1433. Dayton P, Morgan K, Klibanov A, Brandenburger G, Nightingale K, Ferrara K. A preliminary evaluation of the effects of primary and secondary radiation forces on acoustic contrast agents. 1997; 44(6):1264-1277. Duck F, Physical Properties of Tissue. Harcourt Brace Jovanovich, San Diego. 1990. Fowlkes J, Abramowicz J, Church C, Holland C, Miller D, O'Brien W, Sanghvi N, Stratmeyer M, Zachary J. American institute of ultrasound in medicine consensus report on potential bioeffects of diagnostic ultrsound: Executive summary. Journal of Ultrasound in Medicine. 2008; 27:503-515. Goertz D, Frinlink M, Bouakaz A, Chin C, de Jong N, van der Steen A. The effect of bubble size on nonlinear scattering from microbubbles at high frequencies. 2003 IEEE Ultrasonics Symposium. 2003:1503-1506. Hashimoto M, Ueno T, Iriki M. What roles does the organum vasculosum laminae terminalis play in fever in rabbits? Pfl″ugers Arch Eur J. Physiol. 1994; 429:50-57. Hynynen K, McDannold N, Martin H, Jolesz F, Vykhodtseva N. The threshold for brain damage in rabbits induced by bursts of utlrasound in the presence of an ultrasound contrast agent (OtisonR). 2003; 29(3):473-481. Hynynen K, McDannold N, Sheikov N, Jolesz F, Vykhodtseva N. Local and reversible blood-brain barrier disruption by noninvasive focused ultrasound at frequencies suitable for trans-skull sonications. Neuroimage. 2005; 24:12-20. Hynynen K, McDannold N, Vykhodtseva N, Jolesz F. Noninvasive MR imaging-guided focal opening of the blood-brain barrier in rabbits. Radiology. 2001; 220:640-646. Jakobsen J, Oyen R, Thomsen H, Morcos S. Safety of ultrasound contrast agents. Eur 24 Radiol. 2005; 15:941-945. Kinoshita M, McDannold N, Jolesz F, Hynynen K. Noninvasive localized delivery of herceptin to the mouse brain by MRI-guided focused ultrasound-induced blood-brain barrier disruption. Proceedings of the National Academy of Sciences. 2006a; 103(31):11719-11723. Kinoshita M, McDannold N, Jolesz F, Hynynen K. Targeted delivery of antibodies through the blood-brain barrier in MRI-guided focused ultrasound. Biochemical and Biophysical Research Communications. 2006b; 340:1085-1090. McDannold N, Vykhodtseva N, Hynynen K. Targeted disruption of the blood-brain barrier with focused ultrasound: association with cavitation activity. 2006; 51:793-807. McDannold N, Vykhodtseva N, Hynynen K. Use of ultrasound pulses combined with Definity for targeted blood-brain barrier disruption: A feasibility study. Ultrasound in Med. & Biol. 2007; 33(4):584-590. McDannold N, Vykhodtseva N, Hynynen K. Blood-brain barrier disruption induced by focused ultrasound and circulating preformed microbubbles appears to be characterized by the mechanical index. 2008a; 34(5):834-840. McDannold N, Vykhodtseva N, Hynynen K. Effects of acoustic parameters and ultrasound contrast agent dose on focused-ultrasound induced blood-brain barrier disruption. 2008b; 34(6):930-937. McDannold N, Vykhodtseva N, Jolesz F, Hynynen K. MRI investigation of the threshold for thermally induced blood-brain barrier disruption and brain tissue damage in the rabbit brain. Magnetic Resonance in Medicine. 2004; 51:913-923. McKinley M, Allen A, Burns P, Colvill L, Oldfield B. Interaction of circulating hormones with the brain: The roles of the subformical organ and the organum vasculosum of the lamina terminalis. Clinical and Experimental Pharmacology and Physiology. 1998; 25(S1):S61-S67. Mesiwala A, Farrel L, Wenzel H, Silbergeld D, Crum L, Winn H, Mourad P. High-intensity focused ultrasound selectively disrupts the blood-brain barrier in vivo. Ultrasound Med. Biol. 2002; 28:389-400. Nock L, Trahey G, Smith S. Phase aberration correction in medical ultrasound using speckle brightness as a quality factor. J. Acoust. Soc. Am. 1989; 85(5):1819-1833. Price C, Hoyda T, Ferguson A. The area postrema: A brain monitor and integrator of systemic autonomic state. The Neuroscientist. 2008; 14(2):182-194. Raymond S, Skoch J, Hynynen K, Bacskai B. Multiphoton imaging of ultrasound/Optison mediated cerebrovascular effects in vivo. Journal of Cerebral Blood Flow &Metabolism. 2007; 27:393-403. Raymond S, Treat L, Dewey J, McDannold N, Hynynen K, Bacskai B. Ultrasound enhanced delivery of molecular imaging and therapeutic agents in Alzheimer's disease mouse models. PLoS One. 2008; 3(5):1-7. Reinhard M, Hetzel A, Kruger S, Kretzer S, Talazko J, Ziyeh S, Weber J, Els T. Bloodbrain barrier disruption by low-frequency ultrasound. Stroke. 2006; 37:1546-1548. Sassaroli E, Hynynen K. Forced linear oscillations of microbubbles in blood capillaries. J. Acoust. Soc. Am. 2004; 115(6):3235-3243. Sassaroli E, Hynynen K. Resonance frequency of microbubbles in small blood vessels: a numerical study. 2005; 50:5293-5305. Schlachetzki F, Holscher T, Koch H, Draganski B, May A, Schuierer G, Bogdahn U. Observation on the integrity of the blood-brain barrier after microbubble destruction by diagnostic transcranial color-coded sonography. J Ultrasound Med. 2002; 21:419-429. Sheikov N, McDannold N, Vykhodtseva N, Jolesz F, Hynynen K. Cellular mechanisms of the blood-brain barrier opening induced by ultrasound in presence of microbubbles. Ultrasound in Med. & Biol. 2004; 30(7):979-989. Shimamura M, Sato N, Taniyama Y, Yamamoto S, Endoh M, Kurinami H, Aoki M, Ogihara T, Kaneda Y, Morishita R. Development of efficient plasmid DNA transfer into adult rat central nervous system using microbubble-enhanced ultrasound. Gene Therapy. 2004; 11:1532-1539. Tanter M, Thomas J, Fink M. Focusing and steering through absorbing and aberrating layers: Application to ultrasonic propagation through the skull. Journal of the Acoustical Society of America. 1998; 103(5):2403-2410. Tran T, Le Guennec J, Bougnoux P, Tranquart F, Bouakaz A. Characterization of cell membrane response to ultrasound activated microbubbles. IEEE Transactions on Ultrasonics, Ferroelectrics, and Frequency Control. 2008; 55(1):44-49. Unger E, Porter T, Culp W, Labell R, Matsunaga T, Zutshi R. Therapeutic applications of lipid-coated microbubbles. Advanced Drug Delivery Reviews. 2004; 56(9):1291-1314.

We claim:

1. A method of opening a blood-brain barrier of a subject comprising the steps of:
(a) administering a microbubble agent into the bloodstream of said subject, and
(b) applying either (i) an unfocused ultrasound to the whole brain of said subject to open the blood brain barrier in the whole brain, or (ii) an electronically focused ultrasound beam to a portion of the brain.

2. The method of claim 1 wherein said step (a) is performed before step (b) or at the same time as step (b).

3. The method of claim 1 wherein said step (a) is performed within 15 minutes, within 10 minutes, within 5 minutes, within 3 minutes or within one minute of step (b).

4. The method of claim 1 wherein said subject is a mammal.

5. The method of claim 1 wherein said microbubble agent is a lipid-type microspheres injectable suspension or a protein-type microspheres injectable suspension.

6. The method of claim 1 wherein said microbubble agent is selected from the group consisting of: an octafluoropropane/albumin agent (Optison), a perflutren lipid microsphere agent (Definity), a galactose-palmitic acid microbubble suspension agent (Levovist), an air/albumin agent (Albunex and Quantison), an air/palmitic acid agent (Levovist/SHU508A), a perfluoropropane/phospholipids agent (MRX115, DMP115), a dodecafluoropentane/surfactant agent (Echogen/QW3600), a perfluorobutane/albumin agent (perfluorocarbon exposed sonicated dextrose albumin), a perfluorocarbon/surfactant agent (QW7437), a perfluorohexane/surfactant agent (Imagent/AFO150), a sulphur hexafluoride/phospholipids agent (Sonovue/BR1), a perfluorobutane/phospholipids agent (BR14), an air/cyanoacrylate agent (Sonavist/SHU563A), and a perfluorocarbon/surfactant agent (Sonazoid/NC100100).

7. The method of claim 1 further comprising a step of administering a diagnostic or therapeutic agent to the blood of the subject before step (b) or within 4 hours of step (b).

8. The method of claim 7 wherein said therapeutic agent is selected from the group consisting of a chemotherapeutic agent, a neurotherapeutic agent, and a combination thereof.

9. The method of claim 1 wherein said applying step for the delivery of ultrasound comprises the delivery of ultrasound from an ultrasound source through a fluid coupler applied directly to the head of the subject.

10. The method of claim 9 wherein the fluid coupler may be applied to only one side of the subject's head.

11. The method of claim 1, wherein said step of applying ultrasound to the brain comprises applying ultrasound to a surgically created window in the skull through the fluid coupler being in contact with the window.

12. The method of claim 1 wherein the ultrasound may be generated by an unfocused ultrasound transducer or a phased array ultrasound transducer.

13. The method of claim 12 wherein the phased array ultrasound transducer is a diagnostic phased array transducer.

14. The method of claim 12 wherein the ultrasound transducer may have an output frequency of between 0.1 to 10 MHz.

15. The method of claim 12 wherein the ultrasound may be applied for a time between 10 milliseconds to 10 minutes.

16. The method of claim 12 wherein the ultrasound is applied continuously or applied in a burst mode.

17. The method of claim 16 wherein the burst mode has a repetition frequency of between 10 Hz to 100 kHz and burst lengths of 2 microseconds to 100 milliseconds.

18. The method of claim 9 wherein the fluid coupler comprises a contained volume of fluid.

19. The method of claim 18 wherein the fluid is selected from the group consisting of water, ultrasonic gel, or a substance of comparable acoustic impedance.

20. The method of claim 18 wherein the fluid may be contained in a fluid cylinder with at least a flexible end portion that conforms to the subject's head.

21. A method for providing an imaging contrast agent to the whole brain comprising the steps of
(a) administering a microbubble agent into the bloodstream of said subject;
(b) administering an imaging contrast agent into the bloodstream of said subject; and
(c) applying an unfocused ultrasound to the whole brain of said subject to open the blood brain barrier to allow the contrast agent to cross the blood brain barrier, wherein step (b) is performed before said steps (a), before said step (c) or within 4 hours after said step (c).

22. The method of claim 21 wherein the image contrast agent is selected from the group consisting magnetic resonance contrast agents, x-ray contrast agents (and x-ray computed tomography), optical contrast agents, positron emission tomography (PET) contrast agents, single photon emission computer tomography (SPECT) contrast agents, and molecular imaging agents.

23. The method of claim 21 wherein the imaging contrast agent is selected from the group consisting of gadopentetate dimeglumine, Gadodiamide, Gadoteridol, gadobenate dimeglumine, gadoversetamide, iopromide, Iopamidol, Ioversol, or Iodixanol, and Iobitridol.
